(19) United States
(12) Patent Application Publication
(10) Pub. No.: US 2005/0165232 A1
(43) Pub. Date: Jul. 28, 2005

(54) Phenyl substituted imidaopyridines and phenyl substituted benzimidazoles

(76) Inventors: Richard Beresis, Matawan, NJ (US); Steven L. Colletti, Princeton Junction, NJ (US); James Burke Doherty, Montvale, NJ (US); Dennis M. Zaller, Scotch Plains, NJ (US)

Correspondence Address: MERCK AND CO., INC, P O BOX 2000, RAHWAY, NJ, 07065-0907, US

(21) Appl. No.: 10/513,932

(22) PCT Filed: May 9, 2003
(86) PCT No.: PCT/US03/14777

Related U.S. Application Data
(60) Provisional application No. 60/378,028, filed on May 13, 2002.

(51) Int. Cl. C07D 43/02; A61K 31/501

(52) U.S. Cl. 544/238; 514/252.06

(57) ABSTRACT

Compounds described by the formula (I) or formula (II): (I), (II), or pharmaceutically acceptable salts thereof, are inhibitors of p38 useful in the treatment of inflammatory diseases such as arthritis. Compounds may be selective adenosine A1 antagonists useful in the treatment of neurological disorders such as dementia and depression.

### BACKGROUND OF THE INVENTION

Mitogen-activated protein (“MAP”) kinases mediate the surface-to-nucleus signal transduction in a cell. Protein kinases that activate and phosphorylate MAP are known as mitogen-activated protein kinase kinases (“MKK”). One such MKK specifically phosphorylates and activates the p38 MAP kinase (“p38”) and is called MKK3. U.S. Pat. Nos. 5,736,381 and 5,804,427 describe human mitogen-activated kinase kinase isoforms. International Publication No. 98/00539 describes a human gene encoding an M3-Interacting Protein.

Xia et al., Science, 270:1326-1331(1995) describes the p38 signal transduction pathway as being activated by proinflammatory cytokines and environmental stress. MKK3 is described as being involved in transducing stress signals such as nerve growth factor mediated apoptosis in PC12 cells. It is believed that inhibition of p38 activity can provide relief from acute and chronic inflammation by blocking production of cytokines such as IL-1 and TNF, thereby inhibiting the production of proinflammatory cytokines such as IL-6 and IL-8. In particular, it is believed that p38 inhibitors block the synthesis of TNFα and IL-1β, cytokines, thereby providing relief from inflammatory diseases such as rheumatoid arthritis. Accordingly, it would be desirable to provide novel compounds that are selective and potent inhibitors of the action of p38.

International Publication No. 97/22704 describes the mitogen-activated protein kinase kinase MEK6, which can stimulate phosphorylation and activation of p38 substrates. International Publication Nos. 95/31451, 99/00357 and 98/27098 describe various inhibitors of p38. Nonetheless, there remains a great need to develop inhibitors of the action of p38 for various pharmaceutical and therapeutic applications.

The following reviews describe the biochemistry of adenosine receptor modulation and the application to neuropharmacology: Guieu, et al., Gen. Pharmac. 31:553-561(1998), Poulsen and Quinn, Bioorg. Med. Chem. 6:619-641(1998) and Williams, Nucleosides Nucleotides 10:1087-1099(1991). Adenosine G-protein coupled receptors are located at the synapses of neurons and on dendrites, and the adenosine A₁ subtype is primarily distributed in brain tissue. Endogenous adenosine is known to inhibit the release of many neurotransmitters, excitatory amino acids and hormones. This phenomenon occurs through the GPCR-mediated blockade of calcium ion channel effectors, decreasing the influx of calcium into cells of the central or peripheral nervous system. Antagonism of this sedative effect serves to increase the levels of neurotransmitters such as acetylcholine, dopamine, serotonin, GABA and glutamate, several of which have been successfully targeted in the treatment of neurological disorders by their upregulation. In particular, for instance, adenosine Al antagonism is believed to enhance cognition by the upregulation of acetylcholine and glutamate, and therefore may have therapeutic application to dementias such as Alzheimer's disease. Accordingly, it would be desirable to provide novel compounds that are selective and potent antagonists of the action of adenosine with application to neuroscience pharmacology.

International Publication Nos. 01/39777 and 01/40230 describe various adenosine antagonists with minimal A₁ subtype selectivity. Nonetheless, there remains a great need to develop selective adenosine antagonists for various pharmaceutical and therapeutic applications.

### SUMMARY OF THE INVENTION

The present invention relates to compounds of the following Formula (I) or (II): 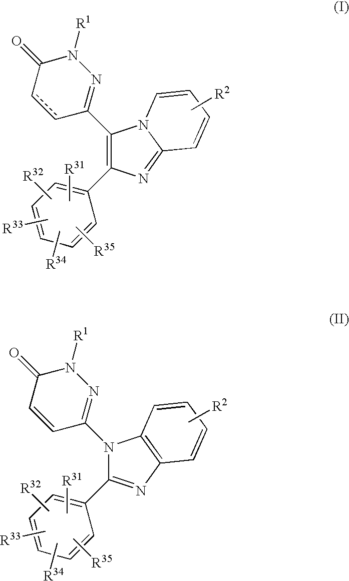
or a pharmaceutically acceptable salt and/or hydrate thereof,

### DETAILED DESCRIPTION OF THE INVENTION

The present invention is directed to compounds represented by Formula (I) or Formula (II): 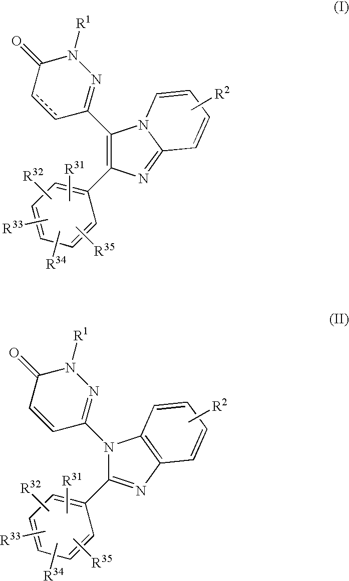
or a pharmaceutically acceptable salt or hydrate thereof, wherein
the dotted line indicates an optional bond;
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl)-N(C₀₋₄alkyl)(C₀₋₄alkyl), C₁₋₄alkyl, C₁₋₆alkoxy, C₁₋₆alkyl-C(O)—C₀₋₄alkyl-, or halogen;
R² is hydrogen, —C(O)—N₃, —NCO, C₁₋₆alkyl- group, —C(O)(C₀₋₄alkyl) group, —(C₀₋₄alkyl)-N(C₀₋₄alkyl)(C₀₋₄alkyl) group, —(C₀₋₄alkyl)-S(O)ₙ—(C₀₋₄alkyl) group, —S(O)₂—N(C₀₋₄alkyl)(C₀₋₄alkyl) group, —C(O)—N(C₀₋₄alkyl)(C₀₋₄alkyl) group, —N(C₀₋₄alkyl)-C(O)—N(C₀₋₄alkyl)(C₀₋₄alkyl) group, —O—C(O)—N(C₀₋₄alkyl)(C₀₋₄alkyl) group, —C(O)—O—(C₀₋₄alkyl) group, —C₀₋₆alkyl-N(C₀₋₄alkyl)-S(O)₂—(C₀₋₄alkyl) group, or —C₀₋₆alkyl-N(C₀₋₄alkyl)-S(O)₂—C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C₁₋₆alkoxy, C₁₋₆alkyl-CO—C₀₋₄alkyl-, or halogen;
R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1, or 2; and
any alkyl is optionally substituted with 1-6 independent halogen.

In one aspect, the present invention is directed to compounds represented by Formula (I): 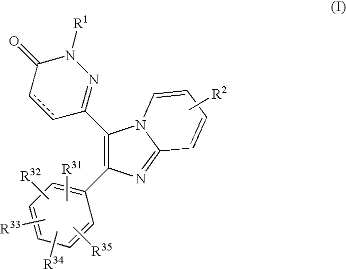
or a pharmaceutically acceptable salt or hydrate thereof, wherein
the dotted line indicates an optional bond;
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl)-N(C₀₋₄alkyl)(C₀₋₄alkyl), C₁₋₄alkyl, C₁₋₆alkoxy, C₁₋₆alkyl-C(O—C₀₋₄alkyl-, or halogen;
R² is hydrogen, —C(O)—N₃, —NCO, C₁₋₆alkyl- group, —C(O)(C₀₋₄alkyl) group, —(C₀₋₄alkyl)-N(C₀₋₄alkyl)(C₀₋₄alkyl) group, —C₀₋₄alkyl)-S(O)ₙ—(C₀₋₄alkyl) group, —S(O)₂—N(C₀₋₄alkyl)(C₀₋₄alkyl) group, —C(O)—N(C₀₋₄alkyl)(C₀₋₄alkyl) group, —N(C₀₋₄alkyl)-C(O)—N(C₀₋₄alkyl)(C₀₋₄alkyl) group, —O—C(O)—N(C₀₋₄alkyl)(C₀₋₄alkyl) group, —C(O)—O—C₀₋₄alkyl) group, —C₀₋₆alkyl-N(C₀₋₄alkyl)-S(O)₂—C₀₋₄alkyl) group, or —C₀₋₆alkyl-N(C₀₋₄alkyl)-S(O)₂—C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C₁₋₆alkoxy, C₁₋₆alkyl-CO—C₀₋₄alkyl-, or halogen;

R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1, or2; and
any alkyl is optionally substituted with 1-6 independent halogen.

In an embodiment of this one aspect, the present invention is directed to compounds represented by: 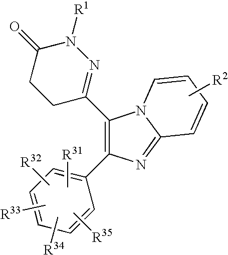
or a pharmaceutically acceptable salt or hydrate thereof, wherein
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl)-N(C₀₋₄alkyl)(C₀₋₄alkyl), C₁₋₄alkyl, C₁₋₆alkoxy, C₁₋₆alkyl-C(O)—C₀₋₄alkyl-, or halogen;
R² is hydrogen, —C(O)—N₃, —NCO, C₁₋₆alkyl- group, —C(O)(C₀₋₄alkyl) group, —(C₀₋₄alkyl)-N(C₀₋₄alkyl)(C₀₋₄alkyl) group, —C₀₋₄alkyl)-S(O)ₙ—(C₀₋₄alkyl) group, —S(O)₂—N(C₀₋₄alkyl)(C₀₋₄alkyl) group, —C(O)—N(C₀₋₄alkyl)(C₀₋₄alkyl) group, —N(C₀₋₄alkyl)-C(O)—N(C₀₋₄alkyl)(C₀₋₄alkyl) group, —O—C(O)—N(C₀₋₄alkyl)(C₀₋₄alkyl) group, —C(O)—O—(C₀₋₄alkyl) group, —C₀₋₆alkyl-N(C₀₋₄alkyl)-S(O)₂—(C₀₋₄alkyl) group, or —C₀₋₆alkyl-N(C₀₋₄alkyl)-S(O)₂—(C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C₁₋₆alkoxy, C₁₋₆alkyl-CO—C₀₋₄alkyl-, or halogen;
R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1, or2; and
any alkyl is optionally substituted with 1-6 independent halogen.

In another embodiment of this one aspect, the present invention is directed to compounds represented by: 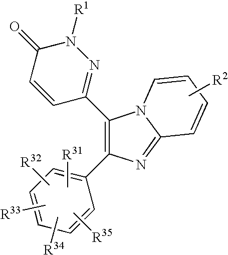
or a pharmaceutically acceptable salt or hydrate thereof, wherein
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl-N(C₀₋₄alkyl)(C₀₋₄alkyl), C₁₋₄alkyl, C₁₋₆alkoxy, C₁₋₆alkyl-C(O)—C₀₋₄alkyl-, or halogen;
R² is hydrogen, —C(O)-N₃, —NCO, C₁₋₆alkyl- group, —C(O)(C₀₄alkyl) group, —(C₀₋₄alkyl)-N(C₀₋₄alkyl)(C₀₋₄alkyl) group, —(C₀₋₄alkyl)-S(O)ₙ—(C₀₋₄alkyl) group, —S(O)₂—N(C₀₋₄alkyl)(C₀₋₄alkyl) group, —C(O)—N(C₀₋₄alkyl)(C₀₋₄alkyl) group, —N(C₀₋₄alkyl)-C(O)—N(C₀₋₄alkyl)(C₀₋₄alkyl) group, —O—C(O)—N(C₀₋₄alkyl)(C₀₋₄alkyl) group, —C(O)—O—C₀₋₄alkyl) group, -C₀₋₆alkyl-N(C₀₋₄alkyl)-S(O)₂—(C₀₋₄alkyl) group, or —C₀₋₆alkyl-N(C₀₋₄alkyl)-S(O)₂—(C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C_{1- 6}alkoxy, C₁₋₆alkyl-CO—C₀₋₄alkyl-, or halogen;
R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1,or2;and
any alkyl is optionally substituted with 1-6 independent halogen.

In a second aspect, the present invention is directed to compounds represented by formula (II): 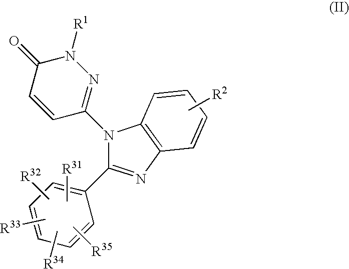
or a pharmaceutically acceptable salt or hydrate thereof, wherein
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl)-N(C₀₋₄alkyl)(C₀₋₄alkyl), C₁₋₄alkyl, C₁₋₆alkoxy, C₁₋₆alkyl-C(O)C₀₋₄alkyl-, or halogen;
R² is hydrogen, —C(O)N₃, —NCO, C₁₋₆alkyl- group, —C(O)(C₀₋₄alkyl) group, —C₀₋₄alkyl)-N(C₀₋₄alkyl)(C₀₋₄alkyl) group, —(C₀₋₄alkyl)-S(O)ₙ—(C₀₋₄alkyl) group, —S(O)₂—N(C₀₋₄alkyl)(C₀₋₄alkyl) group, —C(O)-N(C₀₋₄alkyl)(C₀₋₄alkyl) group, —N(C₀₋₄alkyl)-C(O)—N(C₀₋₄alkyl)(C₀₋₄alkyl) group, —O—C(O)-N(C₀₋₄alkyl)(C₀₋₄alkyl) group, —C(O)—O—(C₀₋₄alkyl) group, —C₀₋₆alkyl-N(C₀₋₄alkyl)-S(O)₂—(C₀₋₄alkyl) group, or —C₀₋₆alkyl-N(C₀₋₄alkyl)-S(O)₂—(C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C₁₋₆alkoxy, C₁₋₆alkyl-CO—C₀₋₄alkyl-, or halogen;
R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1, or 2; and
any alkyl is optionally substituted with 1-6 independent halogen.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “aryl” means an aromatic substituent which is a single ring or multiple rings fused together. When formed of multiple rings, at least one of the constituent rings is aromatic. The preferred aryl substituents are phenyl and naphthyl groups.

The term “cycloalkyloxy” unless specifically stated otherwise includes a cycloalkyl group connected by a short C₁₋₂alkyl length to the oxy connecting atom.

The term “C₀₋₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH—if a bridging group and a —NH₂ if a terminal group. Analogous bridging or terminal groups are clear for an O or S heteroatom.

The term “amine” unless specifically stated otherwise includes primary, secondary and tertiary amines substituted with C₀₋₆alkyl.

The term “carbonyl” unless specifically stated otherwise includes a C₀₋₆alkyl substituent group when the carbonyl is terminal. That is, “carbonyl” means —C(O)—C₀₋₆alkyl unless otherwise stated.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. When a group has an optional substituent, that optional substituent can be on any of the sites readily determined and understood by chemists. That is, for example, a substituent on a cyclopropylC₁₋₄alkyl group can be on the cyclopropyl or on the C₁₋₄alkyl. Further, optionally substituted multiple moieties such as, for example, alkylaryl are intended to mean that the aryl and the alkyl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The present invention includes all such possible isomers as well as mixtures of such isomers unless specifically stated otherwise.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I and II are shown without a definitive stereochemistry at certain positions. The present invention includes all stereoisomers of Formula I and II, and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

Unless specifically stated otherwise or indicated by a bond symbol (dash or double dash), the connecting point to a recited group will be on the right-most stated group. That is, for example, a phenylalkyl group is connected to the main structure through the alkyl and the phenyl is a substituent on the alkyl.

The compounds of the present invention are useful in various pharmaceutically acceptable salt forms. The term “pharmaceutically acceptable salt” refers to those salt forms which would be apparent to the pharmaceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

The pharmaceutically acceptable salts of the compounds of Formula I and II include conventional non-toxic salts or quarternary ammonium salts of the compounds of Formula I and II formed e.g. from non-toxic inorganic or organic acids. For example, non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, sulfanilic, 2-acetoxybenzoic, fumaric, toluenesulfonic, methanesulfonic, ethane disulfonic, oxalic, isethionic, trifluoroacetic and the like.

The pharmaceutically acceptable salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

The compounds of the present invention may have asymmetric centers and occur as racemates, racemic mixtures, and as individual diastereomers. All such isomers, including optical isomers, being included in the present invention.

The invention described herein also includes a pharmaceutical composition which is comprised of a compound described by Formula (I) or (II), or a pharmaceutically acceptable salt thereof, in combination with a pharmaceutically acceptable carrier. The pharmaceutical compositions of the present invention comprise a compound represented by Formula I or II (or pharmaceutically acceptable salts thereof) as an active ingredient, a pharmaceutically acceptable carrier and optionally other therapeutic ingredients or adjuvants. Such additional therapeutic ingredients include, for example, i) Leukotriene receptor antagonists, ii) Leukotriene biosynthesis inhibitors, iii) corticosteroids, iv) H1 receptor antagonists, v) beta 2 adrenoceptor agonists, vi) COX-2 selective inhibitors, vii) statins, viii) non-steroidal anti-inflammatory drugs (“NSAID”), and ix) M2/M3 antagonists.

The invention described herein also includes a method of treating arthritis which is comprised of administering to a mammalian patient in need of such treatment a compound described by Formula (I) or (II), or a pharmaceutically acceptable salt thereof, in an amount which is effective to treat arthritis. The invention includes methods of treating arthritis by administering to a mammalian patient in need of such treatment a compound described by Formula (I) or (II), or a pharmaceutically acceptable salt thereof, in combination or in coadministration with a COX-2 inhibitor.

The invention described herein also includes a method of treating a cytokine mediated disease in a mammal, comprising administering to a mammalian patient in need of such treatment an amount of a compound described by Formula (I) or (II), or a pharmaceutically acceptable salt thereof, in an amount which is effective to treat said cytokine mediated disease.

Of particular interest is a method of treating inflammation in a mammalian patient in need of such treatment, which is comprised of administering to said patient an anti-inflammatory effective amount of a compound described by Formula (I) or (II), or a pharmaceutically acceptable salt thereof.

Another method which is of particular interest is a method of treating a cytokine mediated disease as described herein wherein the disease is osteoporosis.

Another method which is of particular interest is a method of treating a cytokine mediated disease as described herein wherein the disease is non-osteoporotic bone resorption.

Yet another method which is of particular interest is a method of treating a cytokine mediated disease as described herein wherein the disease is Crohn's disease.

This invention also relates to a method of treating arthritis in a mammal in need such treatment, which comprises administering to said mammal an amount of a compound of Formula I or II which is effective for treating arthritis. Such method includes the treatment of rheumatoid and osteoarthritis.

When administered to a patient for the treatment of athritis, the dosage used can be varied depending upon the type of arthritis, the age and general condition of the patient, the particular compound administered, the presence or level of toxicity or adverse effects experienced with the drug, and other factors. A representative example of a suitable dosage range is from as low as about 0.01 mg/kg to as high as about 100 mg/kg. However, the dosage administered is generally left to the discretion of the physician.

This invention also relates to a method of inhibiting the action of p38 in a mammal in need thereof, which comprises administering to said mammal an effective amount of a compound described by Formula (I) or (II), or a pharmaceutically acceptable salt thereof, to inhibit said action of p38, down to normal levels, or in some cases to subnormal levels, so as to ameliorate, prevent or treat the disease state.

The compounds of Formula I or II can be used in the prophylactic or therapeutic treatment of disease states in mammals which are exacerbated or caused by excessive or unregulated cytokines, more specifically IL-1, IL-6, IL-8 or TNF.

The compounds of this invention demonstrates efficacy in the assays described below. Efficacy is shown in the assays by results of less than 10 μM. Advantageously, compounds have results less than 1μM. Even more advantageously, compounds have results less than 0.01 μM. Still more advantageously, compounds have results in the assays of less than 0.01 μM. Because the compounds of Formula I or II inhibit cytokines, such as IL-1, IL-6, IL-8 and TNF, by inhibiting the action of p38 the compounds are useful for treating diseases in which cytokine presence or activity is implicated, such as rheumatoid arthritis, rheumatoid spondylitis, osteoarthritis, gouty arthritis and other arthritic conditions.

The compounds described by Formula (I) or (II), or a pharmaceutically acceptable salt thereof, are also useful to treat other disease states mediated by excessive or unregulated TNF production or activity. Such diseases include, but are not limited to sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, such as osteoporosis, reperfusion injury, graft v. host rejection, allograft rejection, fever, myalgia due to infection, cachexia secondary to infection or malignancy, cachexia secondary to acquired immune deficiency syndrome (AIDS), AIDS, ARC (AIDs related complex), keloid formation, scar tissue formation, Crohn's disease, ulcerative colitis, pyresis, AIDS and other viral infections, such as cytomegalovirus (CMV), influenza virus, and the herpes family of viruses such as Herpes Zoster or Simplex I and II.

The compounds described by Formula (I) or (II), or a pharmaceutically acceptable salt thereof, are also useful topically in the treatment of inflammation such as in the treatment of rheumatoid arthritis, rheumatoid spondylitis, osteoarthritis, gouty arthritis and other arthritic conditions; inflamed joints, eczema, psoriasis or other inflammatory skin conditions such as sunburn; inflammatory eye conditions including conjunctivitis; pyresis, pain and other conditions associated with inflammation.

The compounds described by Formula (I) or (II), or a pharmaceutically acceptable salt thereof, are also useful in treating diseases characterized by excessive IL-8 activity. These disease states include psoriasis, inflammatory bowel disease, asthma, cardiac and renal reperfusion injury, adult respiratory distress syndrome, thrombosis and glomerulonephritis.

The invention thus includes a method of treating psoriasis, inflammatory bowel disease, asthma, cardiac and renal reperfusion injury, adult respiratory distress syndrome, thrombosis and glomerulonephritis, in a mammal in need of such treatment, which comprises administering to said mammal a compound described by Formula (I) or (II), or a pharmaceutically acceptable salt thereof, in an amount which is effective for treating said disease or condition.

When administered to a patient for the treatment of a disease in which a cytokine or cytokines are implicated, the dosage used can be varied depending upon the type of disease, the age and general condition of the patient, the particular compound administered, the presence or level of toxicity or adverse effects experienced with the drug, and other factors. A representative example of a suitable dosage range is from as low as about 0.01 mg/kg to as high as about 100 mg/kg. However, the dosage administered is generally left to the discretion of the physician.

The methods of treatment are preferably carried out by delivering the compound of Formula I or II parenterally. The term ‘parenteral’ as used herein includes intravenous, intramuscular, or intraperitoneal administration. The subcutaneous and intramuscular forms of parenteral administration are generally preferred. The instant invention can also be carried out by delivering the compound of Formula I or II subcutaneously, intranasally, intrarectally, transdermally or intravaginally.

The compounds of Formula I or II may also be administered by inhalation. By ‘inhalation’ is meant intranasal and oral inhalation administration. Appropriate dosage forms for such administration, such as an aerosol formulation or a metered dose inhaler, may be prepared by convention techniques.

The invention also relates to a pharmaceutical composition comprising a compound of Formula I or II and a pharmaceutically acceptable carrier. The compounds of Formula I or II may also be included in pharmaceutical compositions in combination with a second therapeutically active compound.

The pharmaceutical carrier employed may be, for example, either a solid, liquid or gas. Exemples of solid carriers include lactose, terra alba, sucrose, talc, gelatin, agar, pectin, acacia, magnesium stearate, stearic acid and the like. Exemples of liquid carriers are syrup, peanut oil, olive oil, water and the like. Examples of gaseous carriers include carbon dioxide and nitrogen.

Similarly, the carrier or diluent may include time delay material well known in the art, such as glyceryl monostearate or glyceryl distearate, alone or with a wax.

A wide variety of pharmaceutical dosage forms can be employed. If a solid dosage is used for oral administration, the preparation can be in the form of a tablet, hard gelatin capsule, troche or lozenge. The amount of solid carrier will vary widely, but generally will be from about 0.025 mg to about 1 g. When a liquid dosage form is desired for oral administration, the preparation is typically in the form of a syrup, emulsion, soft gelatin capsule, suspension or solution. When a parenteral dosage form is to be employed, the drug may be in solid or liquid form, and may be formulated for administration directly or may be suitable for reconstitution.

Topical dosage forms are also included. Examples of topical dosage forms are solids, liquids and semi-solids. Solids would include dusting powders, poultices and the like. Liquids include solutions, suspensions and emulsions. Semi-solids include creams, ointments, gels and the like.

The amount of a compound of Formula I or II used topically will, of course, vary with the compound chosen, the nature and severity of the condition, and can be varied in accordance with the discretion of the physician. A representative, topical, dose of a compound of Formula I or II is from as low as about 0.01 mg to as high as about 2.0 g, administered one to four, preferably one to two times daily.

The active ingredient may comprise, for topical administration, from about 0.001% to about 10% w/w.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous liquid, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surfactant such as sorbitan esters or polyoxyethylene derivatives thereof. Suspending agents such as natural gums, cellulose derivatives or inorganic materials such as silicas, and other ingredients such as lanolin may also be included.

Assays

Protein Expression and Purification.

Murine p38 containing the FLAG epitope tag was expressed in Drosophila S2 cells under transcriptional control of a copper-inducible metallothionein promoter. Expression of recombinant p38 was induced by treating transfected cells with 1 mM CuSO₄ for 4 hours. To generate active recombinant murine p38, CuSO₄-treated S2 cells were stimulated 10 minutes prior to harvest with 400 mM NaCl, 2 mM Na₃ VO₄, and 100 μg/L okadaic acid. Cell pellets were washed with phosphate-buffered saline, 2 mM Na₃ VO₄, and lysed in 20 mM Tris HCl, pH 7.5, 120 mM NaCl, 1% Triton X-100, 2mM EDTA, 20 mM NaF, 4 mM Na₃ VO₄, 2 mM Prefabloc SC (Boehringer Mannheim). Cell lysates were centrifuged for 10 min at 13,00 ×g, and activated, recombinant murine p38 was immunoaffinity purified from the lysate by column chromatography through anti-FLAG M2 resin (Kodak) that had been equilibrated with lysis buffer. After loading the extract the resin was washed with 10 column volumes of lysis buffer, 10 column volumes buffer A (10 mM Tris HCl, pH 7.5, 500 mM NaCl, 20% glycerol) and 10 column volumes of buffer B (10 mM Tris HCl pH 7.5, 150 mM NaCl, 20% glycerol). The fusion protein was eluted in buffer B containing 100 μg/mL FLAG peptide (Kodak).

The N-terminal 115 amino acids of ATF-2 was expressed in E. coli as a fusion protein with glutathione-S-transferase. The fusion protein was purified over glutathione agarose according to standard procedures (Pharmacia).

p38 Kinase Assay.

p38 kinase assays were performed in a reaction volume of 100 μL in a 96-well plate, at 30° for 45-1200 min under the following conditions: 25 mM Hepes, pH 7.4, 10 mMmgCl₂, 2 mM β-glycerolphosphate, 2 mM DTT, 5 μM ATP, 10 μCi [γ-³³P]-ATP and ˜2 μM GST-ATF2. Serial dilutions of compounds were added to each reaction in 2 μL DMSO. 2 μL of DMSO was added to the last row of each reaction plate as the no inhibitor control for each inhibitor titration. The reaction was terminated with an equal volume of a stop solution containing 100 mM EDTA and 15 mM sodium pyrophosphate. PVDF filter plates (MAIPNOB50, Millipore) were pre-wet with methanol and washed with the stop solution. 50 μL aliquots from a single reaction were applied to the filter under vacuum, and the filter was washed twice with 75 mM phosphoric acid. The filter plates were counted in a scintillation counter (Top Count, Packard) and the percent inhibition at each compound concentration is determined.

TNF-α Release Assay.

Blood was obtained from healthy volunteers by venipuncture using sodium heparin as an anti-coagulant. Peripheral blood mononuclear cells (PBMCs) were isolated using Lymphocyte Separation Medium (ICN) according to manufacturers specifications. Isolated PBMCs were washed 3 times with HBSS and diluted to a density of 2×10⁶ cells/mL in RPMI+5% autologous human serum. 50 μL of the serial dilutions of inhibitor were added to wells of a 96-well tissue culture plate followed by addition of 100 μL of PBMCs and then 50 μL of RPMI complete medium containing 400 ng/mL LPS. A control well of cells without compound but with LPS (maximal stimulation control) and one without compound and without LPS (background control) were included in each titration. The cells were incubated for 16 hours in a humidified incubator at 37° C., 5% CO₂. Supernatants were then harvested and TNF-α levels were quantified by immunoassay using commercial reagents (R&D, Inc).

Human Adenosine A₁ Receptor Binding Assay

Human brain cortex membrane preparations were purchased from ABS, Inc (Wilmington, Del.) and were treated with 2 U/mL adenosine deaminase for 15 min on ice, prior to use. The assay was conducted in Millipore Multiscreen MAFC filter plates (Millipore Corp., MA), using 50 mM Tris/HCl, pH 7.4 as binding buffer. The adenosine A₁ selective antagonist, (“DPCPX“) 3H-cyclopentyl-1,3 -dipropylxanthine, 8-[dipropyl-2,3-3H(N)] (NEN, Boston, Mass.) was used as the radioligand at a final concentration of 0.6 nM. Dilutions of compounds were prepared in DMSO at 100× the desired assay concentration. Typically, final compound concentration ranged from 10 μM -500 pM. Unlabeled 8-cyclopentyl-1,3-dipropylxanthine (Sigma, Saint Louis, Mo.) was titered as a positive control. 100 μg of human cortex membranes was added to each well of the assay and the reaction was allowed to incubate for 1 h at rt. Wells in which inhibitors were omitted served as 0% inhibition. Wells in which 1 μM 8-cyclopentyl-1,3-dipropylxanthine was present served as 100% inhibition. At the end of the incubation period, the plates were filtered and washed twice with 100 μL of ice cold binding buffer. After transfer to adapter plates (Packard, Downers Grove, Ill.), 50 μL Ready Safe scintillation cocktail (Beckman, Fullerton, Calif.) was added. Plates were sealed and placed on a shaker for 1 min, and counted on a Topcount (Packard). Percent inhibition was calculated for each well and IC₅₀ values were determined based on a four parameter fit algorithm.

Alternate Human Adenosine A₁ Receptor Binding Assay

Alternatively, adenosine A₁ radioligand binding was performed as follows. Human recombinant CHO cells were used with 1 nM ³H DPCPX as ligand. The vehicle used was 1% DMSO, the incubation buffer used was 20 mM HEPES, pH 7.4, 10 mM MgCl₂, 100 mM NaCl, and the incubation conditions were 90 min at 25° C. A non-specific ligand (reference compound) 100 μM R(−)-PIA (N6-(R-phenylisopropyl)adenosine) was used, and specific binding was 85%, B=2.7 pmol/mg protein, K_{d}=1.4 nM.

Human Adenosine A_{2A} Receptor Binding Assay

Adenosine A_{2A} radioligand binding was performed as follows. Human recombinant HEK-293 cells were used with 0.05 μM ³H 2-[[p-(2-carboxyethyl)phenethyl]amino]-5′-N-ethylcarboxamidoadenosine (“CGS-21680”) as ligand. The vehicle used was 1% DMSO, the incubation buffer used was 50 mM Tris-HCl, pH 7.4, 10 mM MgCl₂, 1 mM EDTA, 2U/mL adenosine deaminase, and the incubation conditions were 90 min at 25° C. The non-specific ligand (reference compound) used was 50 μM 5′-N-ethylcarboxamidoadenosine (“NECA”), and specific binding was 85%, Bₘₐₓ=7 pmol/mg protein, K_{d}=0.064 μM.

Human Adenosine A_{2B} Receptor Binding Assay

Adenosine A_{2B} radioligand binding was performed as follows. Human recombinant HEK-293 cells were used with 9 nM ³H DPCPX as ligand. The vehicle used was 1% DMSO, the incubation buffer used was 10 mM HEPES, pH 7.4, 1 mM EDTA, 0.1 mM benzamidine, 2U/mL adenosine deaminase, and the incubation conditions were 80 min at 25° C. The non-specific ligand (reference compound) used was 10 μM DPCPX, and specific binding was 60%, Bₘₐₓ=0.96 pmol/mg protein, K_{d}=0.04 μM.

Rat Adenosine A₃ Receptor Binding Assay

Adenosine A₃ radioligand binding was performed as follows. Rat recombinant EBNA cells were used with 1 nM ¹²⁵I AB-MECA as ligand. The vehicle used was 1% DMSO, the incubation buffer used was 50 mM Tris-HCl, pH 7.4, 1 mM EDTA, 10 mM MgCl₂, 1.5 U/mL adenosine deaminase added fresh at the time of assay, and the incubation conditions were 4 h at 25° C. The non-specific ligand (reference compound) used was 100 μM R(−)-PIA, and specific binding was 90%, Bₘₐₓ=1.3 pmol/mg protein, K_{d}=1.3 nM.

Rat Adenosine A₁ Tissue Assay.

An adenosine A₁ tissue assay was performed as follows to determine antagonist versus agonist functional activity. Wistar rat (ca. 275 g) vas deferens were used with the adenosine A₁ agonist reference compound, N6-(cyclohexyl)adenosine (“CHA”) (0.3 μM, 100%) and the adenosine A₁ antagonist reference compound, DPCPX (10 nM, 87%). The vehicle used was 0.1% DMSO, the incubation buffer used was Krebs at pH 7.4, and the incubation time was 5 min at 32° C. The administration volume was 10 μL, the bath volume was 10 mL, and the time of assessment was 5 min using an isometric (gram changes) quantitation method. The criteria for functional agonism was a ≧50% reduction of neurogenic twitch relative to CHA response. The criteria for functional antagonism was a ≧50% inhibition of CHA-induced relaxation.

The compounds of this invention demonstrated efficacy in the above assays by results of less than 10 μM. Advantageous compounds had results less than 1 μM. Even more advantageous compounds had results less than 0.1 μM. Still more advantageous compounds had results in the assays of less than 0.01 μM. In the above assays, compounds of this invention demonstrated significant functional adenosine A₁ antagonism (ca. 60%) in an IC₅₀ range of 1 nM to 100 nM. In the above assays, compounds of this invention demonstrated 500-fold binding selectivity for the adenosine A₁ receptor over the adenosine A_{2A} receptor subtype. In the above assays, compounds of this invention demonstrated >1000-fold binding selectivity for the adenosine A₁ receptor over the A_{2B} and A₃ adenosine receptor subtypes. Certain compounds of this invention demonstrated 20-fold selectivity for p38 kinase inhibition over adenosine A₁ receptor binding in the above assays. Certain compounds of this invention demonstrated a range of 10-fold to 200-fold selectivity for adenosine A₁ receptor binding over p38 kinase inhibition in the above assays.

Thus, the compounds of this invention are effective inhibitors of cytokines—particularly p38 and TNF-alpha. Accordingly, the compounds of this invention are effective to treat inflammation in a mammalian patient in need of such treatment by administering to the patient an anti-inflammatory effective amount of a compound of this invention.

As a result, the compounds of this invention are also effective for treating rheumatoid arthritis, osteoarthritis, endotoxemia, toxic shock syndrome, inflammatory bowel disease, tuberculosis, atherosclerosis, muscle degeneration, cachexia, psoriatic arthritis, Reiter's syndrome, rheumatoid arthritis, gout, traumatic arthritis, rubella arthritis or acute synovitis by administering an effective amount of a compound of this invention.

Further, as a result, the compounds of this invention are also effective for treating rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcosis, bone resorption diseases, reperfusion injury, graft v. host rejection, allograft rejection, fever, myalgia due to infection, cachexia secondary to infection or malignancy, cachexia secondary to acquired immune deficiency syndrome (AIDS), AIDS related complex (ARC), keloid formation, scar tissue formation, Crohn's disease, ulcerative colitis or pyresis by adminstering an effective amount of a compound of this invention.

Further, as a result, the compounds of this invention are effectuve for treating osteoporosis in a mammalian patient in need of such treatment.

Further, as a result, the compounds of this invention are effective for treating bone resorption in a mammalian patient in need of such treatment.

Further, as a result, the compounds of this invention are effective for treating Crohn's disease in a mammalian patient in need of such treatment.

Also as a result, the compounds of this invention are effective for treating neurodegenerative disease, Parkinson's disease, anxiety, psychosis, schizophrenia, and substance abuse.

Further, as a result, the compounds of this invention are effective for treating pain and migraine.

Further, as a result, the compounds of this invention are also effective for treating stroke and cerebrovascular disease.

Further, as a result, the compounds of this invention are effective as antidementia, antidepressant, antianxiety, antipsychotic, anticatalepsy, antiparkinsonian, anxiolytic, nootropic, analgesic, or psychostimulent compounds. The compounds are also effective as a therapeutic for cerebral circulation.

Further, as a result, the compounds of this invention can be used for cognitive enhancement, for their antidepressant action, their cerebral vasodilating action, and for their action of increasing cerebral blood flow.

Furthermore, the selective adenosine A₁ antagonism properties of the compounds of this invention leads to the compounds being effective in the treatment and prevention of depression and dementia (eg. Alzheimer's disease, cerebrovascular dementia, and dementia accompanying Parkinson's disease).

The compounds of the invention are prepared by the following reaction schemes. All substituents are as defined above unless indicated otherwise.

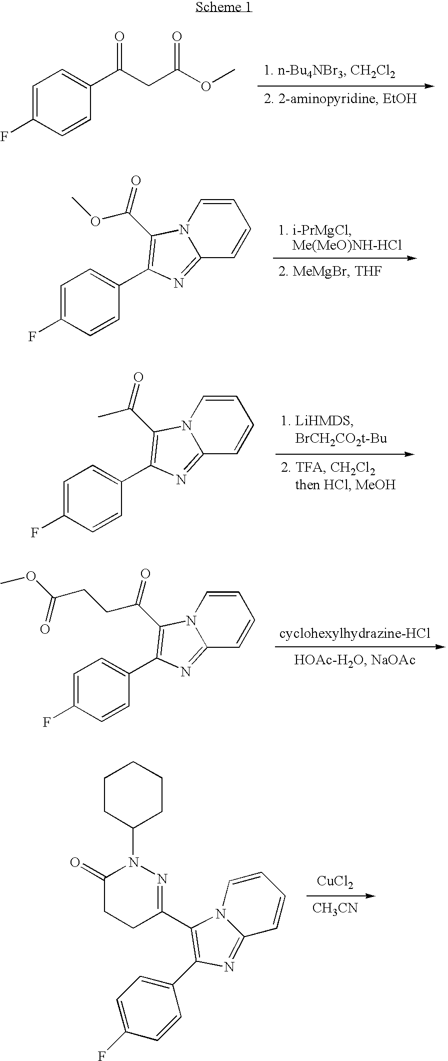

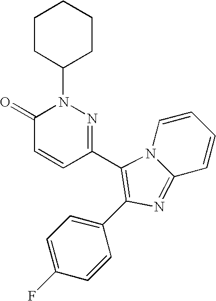

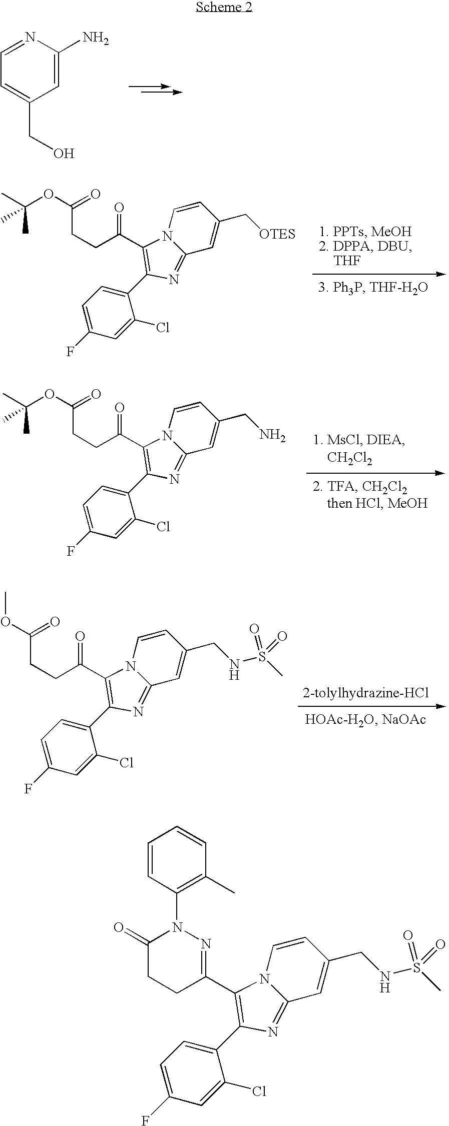

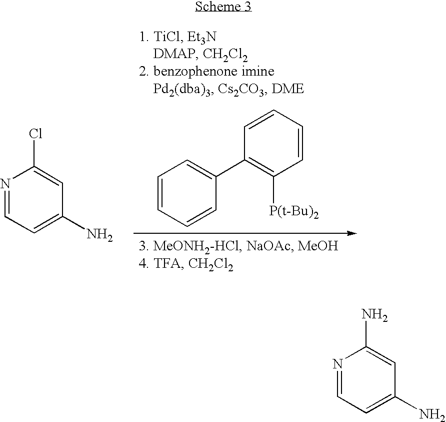

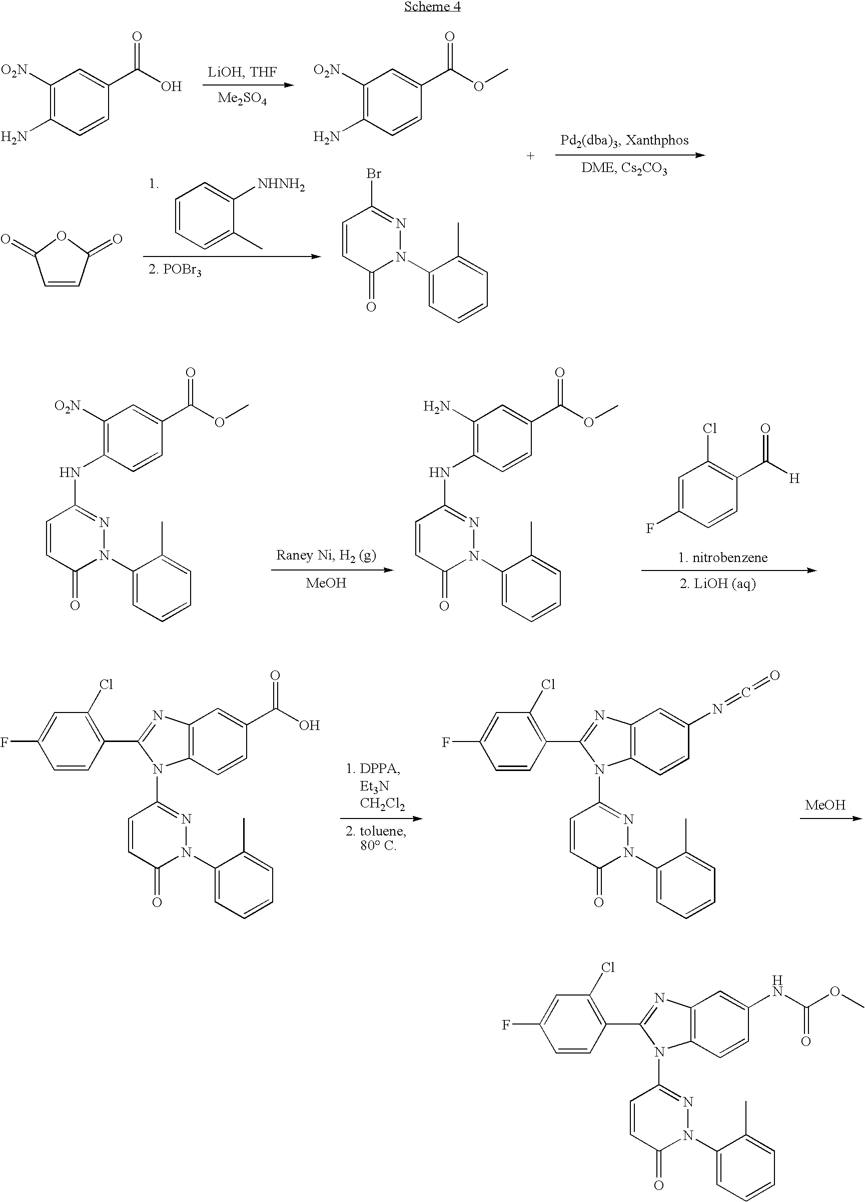

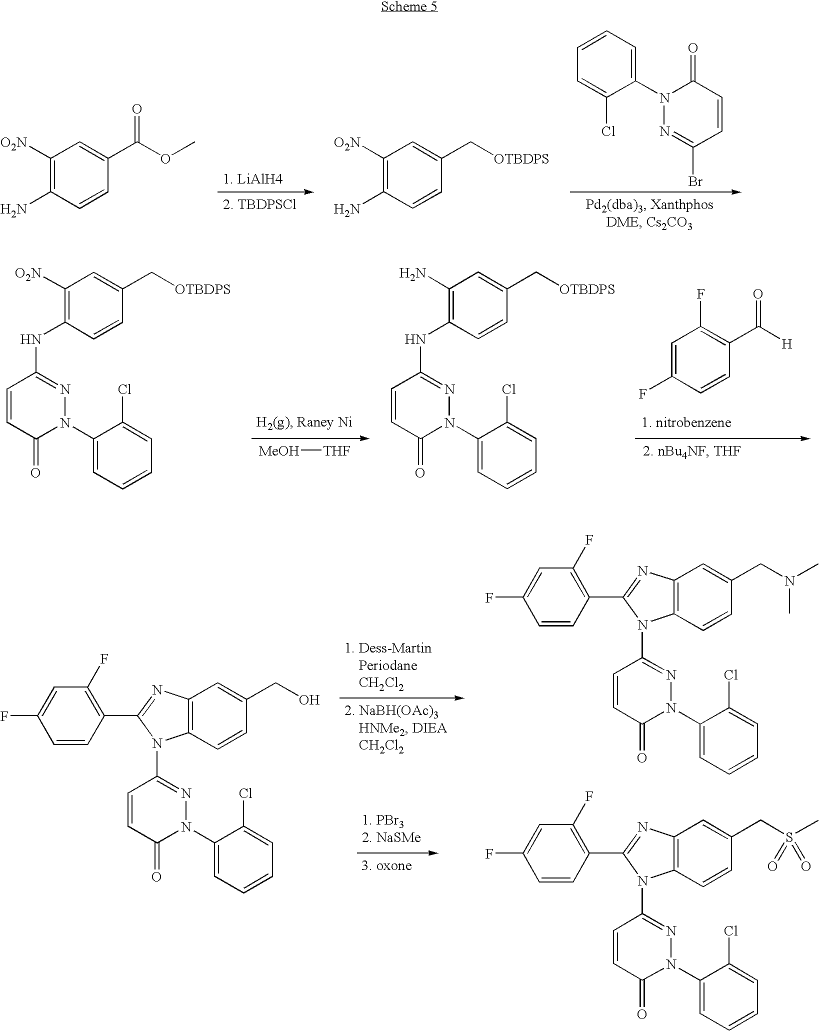

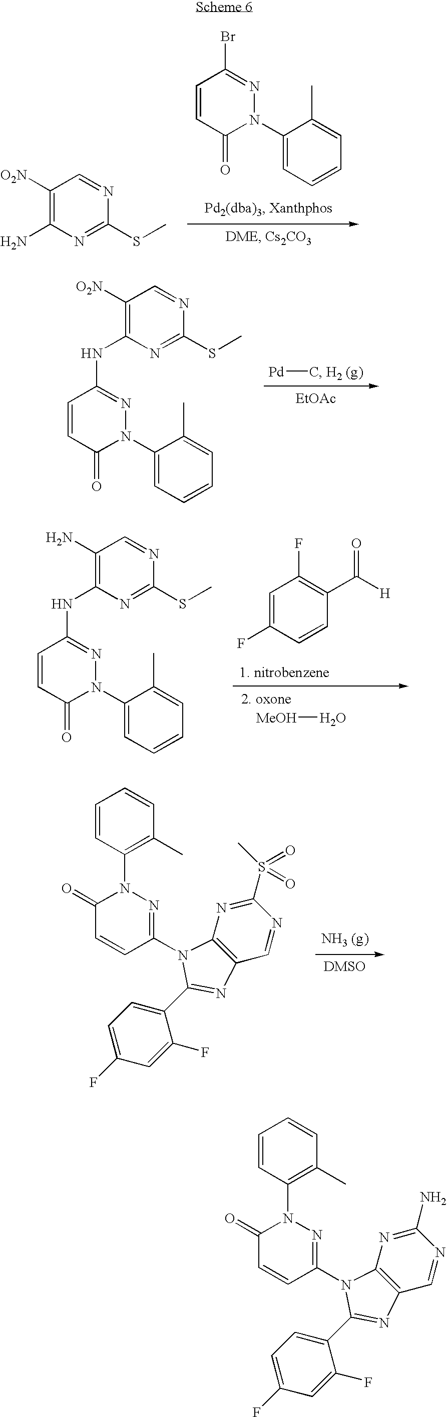

The following examples illustrate the preparation of some of the compounds of the invention and are not to be construed as limiting the invention disclosed herein.

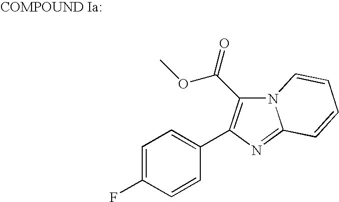

COMPOUND Ia was prepared from the commercially available methyl 4-fluorobenzoyl acetate. To a solution of methyl 4-fluorobenzoyl acetate (10 g, 51.0 mmol) in CH₂Cl₂ (130 mL) at 0° C. was added solid tetrabutylammonium tribromide (26 g, 53.6 mmol). The reaction mixture was maintained at 0° C. for 2 h and then slowly warmed to 23° C. and maintained for 1 h. The orange reaction mixture was then partitioned between NaHCO_{3(aq)} and CH₂Cl₂, the organic phase washed thrice with NaHCO_{3(aq)}, then dried over anhydrous sodium sulfate and concentrated in vacuo. The crude product was vacuum pumped for 30 min, diluted into anhydrous ethanol (250 mL) and treated with commercially available 2-aminopyridine (24 g, 255 mmol). The reaction mixture was warmed to 60° C. and maintained for 14 h, cooled to rt, partitioned between NaHCO_{3(aq)} and CHCl₃, the organic phase dried over anhydrous sodium sulfate and concentrated in vacuo. The crude material was purified by flash chromatography (Biotage 65M, SiO₂, hexane to 20% acetone-hexane gradient elution) to provide COMPOUND Ia which was characterized by ¹H NMR, HPLC and mass spectrometry (m/z: 271 (M⁺+1)).

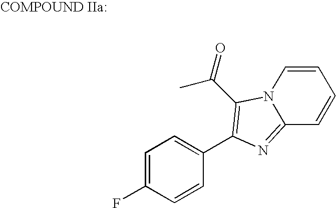

COMPOUND Ia (9.5 g, 35.2 mmol) was diluted into dry THF (176 mL) and N,O-dimethylhydroxylamine hydrochloride (10.3 g, 105.6 mmol) was added. The mixture was cooled to −10° C. and a 2 molar solution of isopropylmagnesium chloride in THF (106 mL, 211.1 mmol) was added under nitrogen. The reaction mixture was maintained at −10° C. for 1 h and then quenched into water. The mixture was then partitioned between NaHCO_{3(aq)} and CH₂Cl₂, the organic phase dried over anhydrous sodium sulfate and concentrated in vacuo. The crude material was purified by flash chromatography (Biotage 65M, SiO₂, 50% ethyl acetate-hexane to ethyl acetate gradient elution) to provide the Weinreb amide product which was characterized by ¹H NMR, HPLC and mass spectrometry (m/z: 300 (M⁺+1)).

This material (7.75 g, 25.9 mmol) was diluted into dry THF (150 mL), cooled to 0° C. and treated with a 3 molar solution of methylmagnesium bromide in diethyl ether (26 mL, 77.8 mmol) under nitrogen. The reaction mixture was maintained at 0° C. for 30 min, quenched into water, partitioned between NaHCO_{3(aq)} and CH₂Cl₂, the organic phase dried over anhydrous sodium sulfate and concentrated in vacuo. The crude material can either be purified by flash chromatography (Biotage, SiO₂, hexane to 30% acetone-hexane gradient elution) or used in the next step without purification. The highly pure methyl ketone COMPOUND IIa was characterized by ¹H NMR, HPLC and mass spectrometry (m/z: 255 (M⁺+1)).

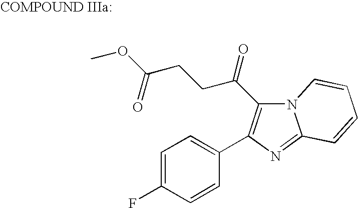

COMPOUND IIa (6.4 g, 25.2 mmol) was diluted into dry THF (250 mL), cooled to −78° C., and treated with a 1 molar solution of lithium bis(trimethylsilyl)amide in THF (38 mL, 37.8 mmol) under nitrogen. The mixture was maintained at −78° C. for 30 min and then treated slowly with t-butyl bromoacetate (19 mL, 125.9 mmol) at −78° C. under nitrogen. The reaction mixture was maintained at −78° C. for 1 h and then slowly warmed to 23° C. over 1 h. The mixture was then quenched into water, partitioned between NaHCO_{3(aq)} and CH₂Cl₂, the organic phase dried over anhydrous sodium sulfate and concentrated in-vacuo. The crude t-butyl ketoester product was then diluted into dry CH₂Cl₂ (180 mL), cooled to 0° C. and treated with trifluoroacetic acid (63 mL). The reaction mixture was maintained at 0° C. for 2 h, warmed to 23° C. for 2 h and then concentrated in vacuo. The crude residue was then diluted into dry methanol, cooled to 0° C., and treated with excess hydrogen chloride gas for 3-5 min. The reaction mixture was maintained at 0° C. for 1 h, and partitioned between NaHCO_{3(aq)} and CHCl₁₃. The organic phase was dried over anhydrous sodium sulfate and concentrated in vacuo. The crude material was purified by flash chromatography (Biotage 65M, SiO₂, hexane to 30% acetone-hexane gradient elution) to provide COMPOUND IIIa which was characterized by ¹H NMR, HPLC and mass spectrometry (m/z: 327 (M⁺+1)).

### EXAMPLE 1

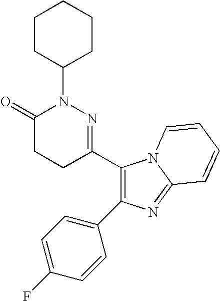

COMPOUND IIIa (32 mg, 0.098 mmol) was combined with sodium acetate (241 mg, 2.94 mmol) and commercially available cyclohexylhydrazine hydrochloride (296 mg, 1.96 mmol). Glacial acetic acid (3.5 mL) and water (1.5 mL) were added, and the reaction mixture was refluxed at 130° C. for 20 h. The mixture was cooled to rt, partitioned between aqueous 2N NaOH and CH₂Cl₂, ensuring an aqueous pH >9, the organic phase dried over anhydrous sodium sulfate and concentrated in vacuo. The crude material was purified by preparative centrifugal thin layer chromatography (Chromatotron, 4 mm SiO₂, 20% to 70% ethyl acetate-hexane gradient elution) to provide semi-pure EXAMPLE 1 which was further purified by preparative reverse phase HPLC (Gilson, YMC C₁₈, 90% H₂O (0.05% TFA)-10% CH₃CN (0.05% TFA) 1 min isocratic then 9 min gradient elution to 100% CH₃CN (0.05% TFA). The product eluent was reduced in volume to aqueous, treated with solid NaHCO_{3(aq)} ensuring an aqueous pH >9, partitioned into CH₂Cl₂ and concentrated in vacuo to provide EXAMPLE 1 which was characterized by ¹H NMR, HPLC and mass spectrometry (m/z: 391 (M⁺+1)).

### EXAMPLES 2-21

The following compounds were prepared under conditions similar to those described above, culminating in the synthesis of EXAMPLE 1. The different groups represented below as R¹ were introduced by the substitution of the appropriate commercially available hydrazine or hydrazine salt in place of cyclohexylhydrazine hydrochloride as shown above in Scheme 1. The different phenyl groups represented below as Ar² were introduced by the substitution of the appropriate β-ketoester (benzoyl acetates were commercially available or prepared by literature methods known to those skilled in the art) in place of methyl 4-fluorobenzoyl acetate as shown in Scheme 1. The following examples were characterized by HPLC and mass spectrometry, and in most cases, additionally by ¹H NMR and/or high resolution mass spectrometry.

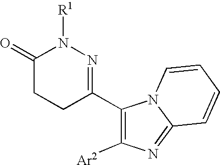

| | | | MS (m/z) |
|---|---|---|---|
| EX. | R^{1 Group} | Ar^{2 Group} | (M⁺ + 1) |
| 2 | 2-Hydroxyethyl | 4-Fluorophenyl | 353 |
| 3 | 2,2,2-Trifluoroethyl | 4-Fluorophenyl | 391 |
| 4 | H | 4-Fluorophenyl | 309 |
| 5 | Benzyl | 4-Fluorophenyl | 399 |
| 6 | Isopropyl | 4-Fluorophenyl | 351 |
| 7 | 2-Chlorophenyl | 4-Fluorophenyl | 419 |
| 8 | 3-Chlorophenyl | 4-Fluorophenyl | 419 |
| 9 | 2-Chlorophenyl | 2-Chlorophenyl | 436 |
| 10 | Cyclohexyl | 2,4-Difluorophenyl | 409 |
| 11 | 2-Chlorophenyl | 3-(Trifluoromethyl)phenyl | 469 |
| 12 | Cyclohexyl | 3-(Trifluoromethyl)phenyl | 441 |
| 13 | 2-Chlorophenyl | 2-Chloro-4--fluorophenyl | 454 |
| 14 | 2,6-Dichlorophenyl | 2-Chloro-4-fluorophenyl | 488 |
| 15 | 2-Tolyl | 2-Chloro-4-fluorophenyl | 433 |
| 16 | 2,6-Dichlorophenyl | 2,3-Dichlorophenyl | 505 |
| 17 | 2-Chlorophenyl | 2,3-Dichlorophenyl | 470 |
| 18 | 2-Tolyl | 2,3-Dichlorophenyl | 450 |
| 19 | 2-(Trifluoromethyl) | 2,3-Dichlorophenyl | 504 |
| | phenyl | | |
| 20 | 2-Tolyl | 2,4-Difluorophenyl | 417 |
| 21 | 2,6-Dichlorophenyl | 2,4-Difluorophenyl | 472 |

### EXAMPLE 22

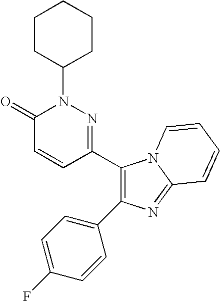

EXAMPLE 1 (2 mg, 0.005 mmol) was combined with copper (II) chloride (34 mg, 0.256 mmol) and diluted into dry acetonitrile (0.2 mL). The reaction mixture was refluxed at 85° C. for 74 h. The mixture was cooled to rt, concentrated in vacuo, partitioned between water and CH₂Cl₂, treated with concentrated ammonium hydroxide, the organic phase dried over anhydrous sodium sulfate and concentrated in vacuo. The crude product was purified by preparative thin layer chromatography (500 micron SiO₂, 20×10 cm, 60% ethyl acetate-hexane) to provide EXAMPLE 22 which was characterized by ¹H NMR, HPLC and mass spectrometry (m/z: 389 (M⁺+1)).

### EXAMPLES 23-37

The following pyridazinones were prepared from their respective dihydropyridazinones under oxidative conditions similar to those described for the synthesis of EXAMPLE 22 as shown in Scheme 1. The following examples were characterized by HPLC and mass spectrometry, and in most cases, additionally by ¹H NMR and/or high resolution mass spectrometry.

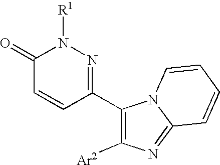

| | | | MS (m/z) |
|---|---|---|---|
| EX. | R¹ Group | Ar² Group | (M⁺ + 1) |
| 23 | 2,2,2-Trifluoroethyl | 4-Fluorophenyl | 389 |
| 24 | 2,6-Dichlorophenyl | 4-Fluorophenyl | 452 |
| 25 | 2-Chlorophenyl | 2-Chlorophenyl | 434 |
| 26 | Cyclohexyl | 2,4-Difluorophenyl | 407 |
| 27 | 2-Chlorophenyl | 3-(Trifluoromethyl)phenyl | 467 |
| 28 | Cyclohexyl | 3-(Trifluoromethyl)phenyl | 439 |
| 29 | 2,6-Dichlorophenyl | 3-(Trifluoromethyl)phenyl | 502 |
| 30 | 2-Chlorophenyl | 2-Chloro-4-fluorophenyl | 452 |
| 31 | 2,6-Dichlorophenyl | 2-Chloro-4-fluorophenyl | 486 |
| 32 | 2-Tolyl | 2-Chloro-4-fluorophenyl | 431 |
| 33 | 2,6-Dichlorophenyl | 2,3-Dichlorophenyl | 503 |
| 34 | 2-Tolyl | 2,3-Dichlorophenyl | 448 |
| 35 | 2-(Trifluoromethyl) | 2,3-Dichlorophenyl | 502 |
| | phenyl | | |
| 36 | 2-Tolyl | 2,4-Difluorophenyl | 415 |
| 37 | 2,6-Dichlorophenyl | 2,4-Difluorophenyl | 470 |

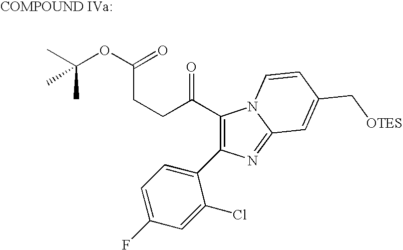

COMPOUND IVa was prepared in an analogous manner to the t-butyl ketoester precursor of COMPOUND IIIa except that 2-amino4-(hydroxymethyl)pyridine (commercially available from CB Research, New Castle, Del.) and 2-chloro4-fluorobenzoyl acetate (see EXAMPLES 2-21) were used in place of 2-aminopyridine and 4-fluorobenzoyl acetate respectively. The product COMPOUND IVa was purified on SiO₂ using 20% ethyl acetate-hexane eluent and characterized by ¹H NMR, HPLC and mass spectrometry (m/z: 547 (M⁺+1)).

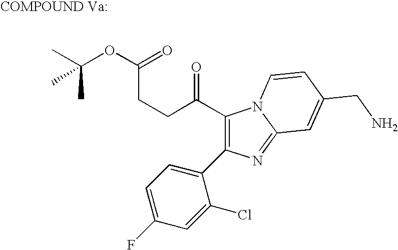

COMPOUND IVa (690 mg, 1.26 mmol) was combined with pyridinium p-toluenesulfonate (1.3 g, 5.18 mmol) and diluted into MeOH (13 mL). The reaction mixture was maintained at 23° C. for 1 h and then concentrated in vacuo. The crude residue was purified by preparative centrifugal thin layer chromatography (Chromatotron, 4 mm SiO₂, hexane to 30% acetone-hexane to 1:9:90 NH₄OH-MeOH—CHCl₃ 3 step gradient elution) to provide 500 mg (92%) of alcohol which was characterized by ¹H NMR, HPLC and mass spectrometry (m/z: 433 (M⁺+1)). This material (200 mg, 0.462 mmol) was diluted into dry THF (8mL), treated with 1,8-diazabicyclo[5.4.0]undec-7-ene (0.065 mL, 0.647 mmol) followed by diphenylphosphoryl azide (0.119 mL, 0.554 mmol), and the reaction mixture was maintained at 23° C. for 14 h. The reaction mixture was partitioned between NaHCO_{3(aq)} and CH₂Cl₂, the organic phase dried over anhydrous sodium sulfate and concentrated in vacuo. The crude residue was purified by preparative centrifugal thin layer chromatography (Chromatotron, 2 mm SiO₂, 20% ethyl acetate-hexane isocratic elution) to provide 140 mg (66%) of azide which was characterized by ¹H NMR. This material (140 mg, 0.306 mmol) was diluted into THF (8 mL), treated with water (0.220 mL, 12.2 mmol) followed by triphenylphosphine (240 mg, 0.918 mmol), and the reaction mixture was maintained at 23 ° C. for 14 h. The reaction mixture was partitioned between NaHCO_{3(aq)} and 30% isopropanol-chloroform, the organic phase dried over anhydrous sodium sulfate and concentrated in vacuo. The crude residue was purified by preparative centrifugal thin layer chromatography (Chromatotron, 2 mm SiO₂, chloroform to 20% methanol-chloroform to 50% methanol-chloroform to 1:9:90 NH₄OH-MeOH—CHCl₃ 4 step gradient elution) to provide COMPOUND Va which was characterized by ¹H NMR, HPLC and mass spectrometry (m/z: 432 (M⁺+1)).

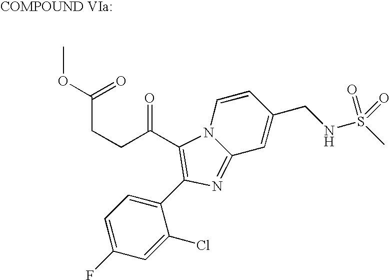

COMPOUND Va (29 mg, 0.067 mmol) was diluted into CH₂Cl₂ (1.3 mL), treated with diisopropylethylamine (0.035 mL, 0.198 mmol), cooled to 0° C., and methanesulfonyl chloride (0.008 mL, 0.099 mmol) added under nitrogen. The reaction mixture was maintained at 0° C. for 3 h, partitioned between NaHCO_{3(aq)} and CH₂Cl₂, the organic phase dried over anhydrous sodium sulfate and concentrated in vacuo. The crude residue was purified by preparative reverse phase HPLC (Gilson, YMC C₁₈, 90% H₂O (0.05% TFA) - 10% CH₃CN (0.05% TFA) 1 min isocratic then 9 min gradient elution to 100% CH₃CN (0.05% TFA). The product eluent was reduced in volume to aqueous, treated with solid NaHCO_{3(aq)} ensuring an aqueous pH >9, partitioned into CH₂Cl₂ and concentrated in vacuo to provide the sulfonamide t-butyl ketoester which was characterized by ¹H NMR, HPLC and mass spectrometry (m/z: 510 (M⁺+1)). This material (7 mg, 0.0138 mmol) was then diluted into dry CH₂Cl₂ (0.140 mL), cooled to 0° C. and treated with trifluoroacetic acid (0.140 mL). The reaction mixture was maintained at 0° C. for 2 h, warmed to 23° C. for 2 h and then concentrated in vacuo. The crude residue was then diluted into dry methanol, cooled to 0° C., and treated with excess hydrogen chloride gas for 1 min. The reaction mixture was maintained at 0° C. for 1 h, partitioned between NaHCO_{3(aq)} and CHCl₃, the organic phase dried over anhydrous sodium sulfate and concentrated in vacuo. The crude product sulfonamide methyl ketoester COMPOUND VIa was used directly in the next cyclization reaction.

### EXAMPLE 38

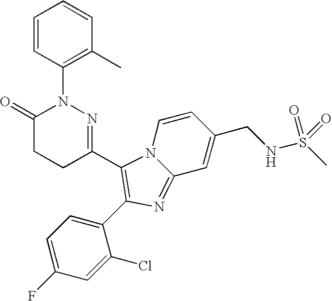

Crude COMPOUND VIa (6 mg, 0.0138 mmol) was combined with sodium acetate (36 mg, 0.435 mmol) and 2-tolylhydrazine hydrochloride (46 mg, 0.29 mmol). Glacial acetic acid (1 mL) and water (0.2 mL) were added, and the reaction mixture was refluxed at 150° C. for 16 h. The mixture was cooled to rt, partitioned between aqueous 2N NaOH and CH₂Cl₂ ensuring an aqueous pH >9, the organic phase dried over anhydrous sodium sulfate and concentrated in vacuo. The crude material was purified by preparative thin layer chromatography (SiO₂, 250 micron, 20×20 cm, 30% THF-hexane then 1:3:96 NH₄OH-MeOH—CHCl₃ then acetonitrile, 3 step gradient elution) to provide EXAMPLE 38 which was characterized by ¹H NMR, HPLC and mass spectrometry (m/z: 540 (M⁺+1)).

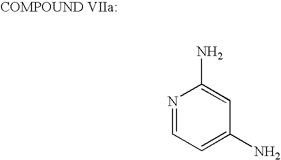

Commercially available 2-chloro4-aminopyridine (5 g, 38.8 mmol) was combined with trityl chloride (14 g, 50.4 mmol), catalytic dimethylaminopyridine (DMAP, 470 mg, 3.88 mmol), diluted into dry methylene chloride (130 mL) and treated with triethylamine (17 mL, 116.3 mmol). The reaction mixture was maintained at 23° C. for 15 h, partitioned between NaHCO_{3(aq)} and CHCl₃, the organic phase dried over anhydrous sodium sulfate and concentrated in vacuo. The crude material was purified by plug flash chromatography using a sintered glass funnel with vacuum (SiO₂, 13×13 cm, hexane to 30% ethyl acetate-hexane gradient elution). The 4-trityl-protected amine was characterized by ¹H NMR and HPLC. This material (6 g, 0.016 mol) was combined with cesium carbonate (26.4 g, 0.081 mol), 2-(di-t-butylphosphino)biphenyl (1.9 g, 0.0065 mol), Pd₂(dba)₃ (3 g, 0.0032mol), diluted into dry DME (100 mL) and treated with benzophenone imine (27 mL, 0.162 mol). The reaction mixture was refluxed at 100° C. for 15 h under nitrogen, cooled to rt, partitioned between NaHCO_{3(aq)} and CHCl₃, the organic phase dried over anhydrous sodium sulfate and concentrated in vacuo. The crude intermediate imine adduct was then combined with sodium acetate (27 g, 0.324 mol) and methoxylamine hydrochloride (20 g, 0.243 mol), diluted into dry methanol (160 mL) and maintained at 23° C. for 1 h. The reaction mixture was partitioned between NaHCO_{3(aq)} and CH₂Cl₂, the organic phase dried over anhydrous sodium sulfate and concentrated in vacuo. The crude material was purified by flash chromatography (Biotage 65M, Sio₂, 50% ethyl acetate-hexane then 1:9:90 NH₄OH-MeOH—CHCl₃ 2 step gradient elution) to provide the 4-trityl-protected COMPOUND VIIa which was characterized by ¹H NMR and HPLC. This intermediate (1.5 g, 4.3 mmol) was deprotected by dilution into methylene chloride (14 mL) and treatment with trifluoroacetic acid (4.3 mL) at 23° C. for 2 h. The reaction mixture was then quenched into water, solid sodium chloride and solid sodium bicarbonate were added, the mixture partitioned into 30% isopropanol-chloroform, and the organic phase was dried over anhydrous sodium sulfate and concentrated in vacuo. The crude material was purified by flash chromatography (Biotage 65M, SiO₂, 100% hexane to 100% chloroform gradient elution followed by 1:9:90 and then 3:27:90 NH₄OH-MeOH—CHCl₃ gradient elution) to provide COMPOUND VIIa which was characterized by ¹H NMR and HPLC.

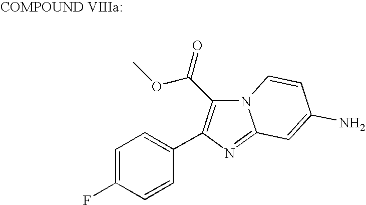

COMPOUND VIIIa was prepared in an analogous manner to COMPOUND Ia except that 1.5 molar equivalents of COMPOUND VIIa was used in place of 5 molar equivalents of 2-aminopyridine, and anhydrous dioxane was substituted for ethanol as the reaction solvent. The reaction mixture was warmed to 60° C. and maintained for 14 h, cooled to rt, partitioned between NaHCO_{3(aq)} and 30% isopropanol-CHCl₃, the aqueous exhaustively extracted, and the combined organic phases dried over anhydrous sodium sulfate and concentrated in vacuo. The crude product was purified by preparative thin layer chromatography (500 micron SiO₂, 20×20 cm, 30% acetone-hexane) to provide COMPOUND VIIIa which was characterized by ¹H NMR. It was anticipated that COMPOUND VIIIa could be transformed into amino-substituted EXAMPLES related to and generated from the chemistry shown in Scheme 1 upon minor synthetic modifications known to those skilled in the art.

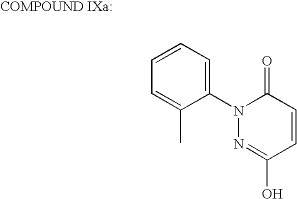

COMPOUND IXa was prepared from commercially available maleic anhydride (9.1 g, 0.093 mol) and ortho-tolylhydrazine hydrochloride (10 g, 0.063 mol) which were combined and diluted into water (154 mL), followed by the addition of concentrated HCl (20 mL). The reaction mixture was refluxed at 110° C. for 15 h, cooled to rt, filtered and the precipitate washed with toluene. The solid product COMPOUND IXa was dried in vacuo and characterized by ¹H NMR, HPLC and mass spectrometry (m/z: 203 (M⁺+1)).

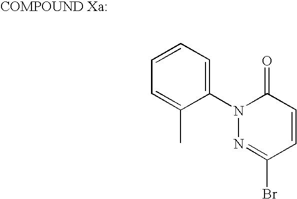

COMPOUND IXa (3 g, 14.85 mmol) was combined with P(O)Br₃ (10 g, 34.84 mmol) in a sealed tube and the reaction mixture heated at 130° C. for 3 h. The mixture was cooled to rt, poured into ice, partitioned between water and ethyl acetate, and the organic phase dried over anhydrous sodium sulfate and concentrated in vacuo. The crude material was purified by flash chromatography (Biotage 65M, SiO₂, hexane to 60% ethyl acetate-hexane gradient elution) to provide the dark orange solid product COMPOUND Xa XIV which was characterized by HPLC and mass spectrometry (m/z: 265 (M⁺+1)).

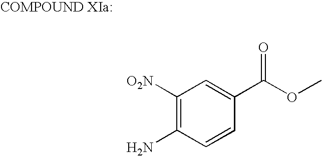

Commercially available 3-nitro-4-aminobenzoic acid (10 g, 54.9 mmol) was diluted into dry THF (133 mL), treated with LiOH—H₂O (670 mg, 27.9 mmol) at 23° C. for 30 min, and dimethyl sulfate (670 mg, 5.3 mmol) was then added. The reaction mixture was heated at 75° C. for 13 h under nitrogen. The mixture was cooled to rt, partitioned between NaHCO_{3(aq)} and ethyl acetate, and the organic phase dried over anhydrous sodium sulfate and concentrated in vacuo. The crude material was purified by flash chromatography (Biotage 65M, SiO₂, hexane to 20% acetone-hexane gradient elution) to provide COMPOUND XIa which was characterized by ¹H NMR, HPLC and mass spectrometry (m/z: 197 (M⁺+1)).

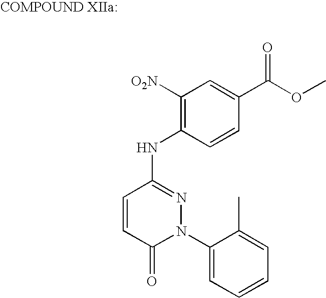

COMPOUND XIa (1 g, 5.1 mmol) was combined with COMPOUND Xa (1 g, 3.77 mmol), cesium carbonate (290 mg, 0.89 mmol), Pd₂(dba)₃ (430 mg, 0.47 mmol), Xanthphos (570 mg, 0.99 mmol) and diluted into dry degassed DME (40 mL). The reaction mixture was refluxed at 90° C. for 20 h under nitrogen in a sealed tube, cooled to rt, filtered and concentrated in vacuo. The crude material was purified by flash chromatography (Biotage 40M, SiO₂, hexane to 20% ethyl acetate-hexane to ethyl acetate gradient elution) to provide the dark yellow solid product COMPOUND XIIa which was characterized by ¹H NMR, HPLC and mass spectrometry (m/z: 381 (M⁺+1)).

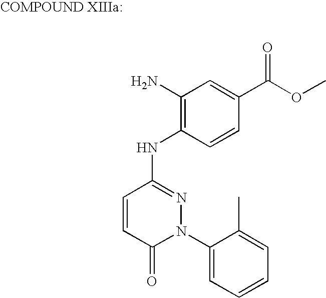

COMPOUND XIIa (600 mg, 1.58 mmol) was diluted into dry methanol (87 mL) and methylene chloride (10 mL), heated to homogeneity, and then the solution was cooled to −30° C. To this cooled reaction mixture was added a methanol solution of catalytic Raney nickel (washed once with water and twice with methanol), a hydrogen atmosphere was introduced via balloon, purged thrice, and the reaction mixture was stirred vigerously at −30° C. for 3 h excluding light. The reaction mixture was filtered through a pad of Celite, quickly washed with methylene chloride and concentrated in vacuo. The crude material was purified by flash chromatography (Biotage 40M, SiO₂, hexane to CH₂Cl₂ to 5% MeOH—CH₂Cl₂ gradient elution) to provide COMPOUND XIIIa which was characterized by ¹H NMR, HPLC and mass spectrometry (m/z: 351 (M⁺+1)).

### EXAMPLE 39

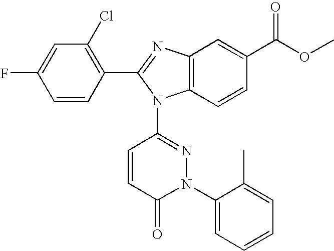

COMPOUND XIIIa (290 mg, 0.83 mmol) was diluted into dry nitrobenzene (21 mL), treated with excess molecular sieves, and 2-chloro4-fluorobenzaldehyde (145 mg, 0.91 mmol) was added. The reaction mixture was heated in a sealed tube at 170° C. for 15 h, and the nitrobenzene was then distilled away at 110° C. The crude product was purified by flash chromatography (Biotage 40M, SiO₂, hexane to CH₂Cl₂ to 5% MeOH—CH₂Cl₂ gradient elution) to provide EXAMPLE 39 which was characterized by HPLC and mass spectrometry (m/z: 489 (M⁺+1)).

### EXAMPLE 40

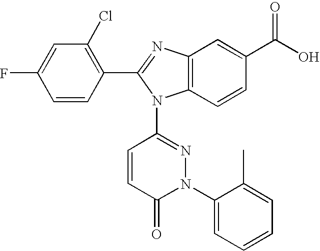

EXAMPLE 39 (250 mg, 0.51 mmol) was diluted into (3:1:1) THF-MeOH—H₂O (5 mL), treated with a 1N solution of aqueous LiOH (2 mL), and the reaction mixture was maintained at 23° C. for 4 h. The reaction mixture was neutralized with a 1N solution of aqueous HCl (2 mL), and chloroform (15 mL) was added. The solution was dried with excess anhydrous sodium sulfate and concentrated in vacuo to provide EXAMPLE 40 which was characterized by HPLC and mass spectrometry (m/z: 475 (M⁺+1)).

### EXAMPLE 41

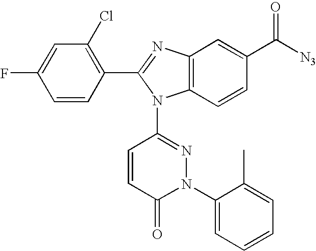

EXAMPLE 40 (50 mg, 0.105 mmol) was diluted into dry CH₂Cl₂ (0.5 mL), treated with triethylamine (0.046 mL, 0.316 mmol), diphenylphosphoryl azide (0.033 mL, 0.158 mmol), and the reaction mixture was maintained at 23° C. for 14 h. The reaction mixture was then purified directly by preparative thin layer chromatography (SiO₂, 1000 micron, 20×20 cm, 50% ethyl acetate-hexane) to provide EXAMPLE 41 which was characterized by HPLC and mass spectrometry (m/z: 500 (M⁺+1)).

### EXAMPLE 42

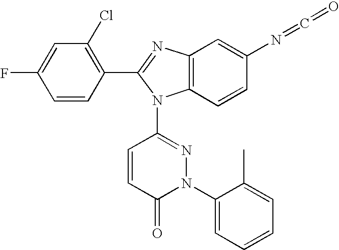

EXAMPLE 41 (20 mg, 0.040 mmol) was diluted into dry toluene (0.8 mL) and the reaction mixture was heated at 80° C. for 4 h. The intermediate isocyanate was characterized by HPLC and mass spectrometry (m/z: 472 (M⁺+1)) directly from the reaction mixture and was not isolated. The isocyanate EXAMPLE 42 was used directly in the next reaction to form carbamate EXAMPLE 43.

### EXAMPLE 43

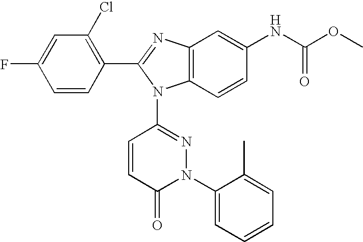

EXAMPLE 42 (5 mg, 0.0106 mmol) in toluene (0.2 mL) was treated with dry MeOH (0.002 mL, 0.053 mmol) and maintained at 23° C. for 14 h. The reaction mixture was then partitioned between NaHCO_{3(aq)} and 30% isopropanol-chloroform, and the organic phase dried over anhydrous sodium sulfate and concentrated in vacuo to provide EXAMPLE 43 which was characterized by ¹H NMR, HPLC and mass spectrometry (m/z: 504 (M⁺+1)).

### EXAMPLE 44

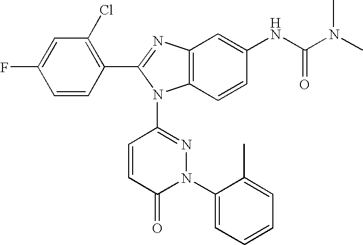

EXAMPLE 44 was prepared as in EXAMPLE 43 by the addition of dimethylamine to isocyanate EXAMPLE 42 in toluene. The purified product was characterized by ¹H NMR, HPLC and mass spectrometry (m/z: 517 (M⁺+1)).

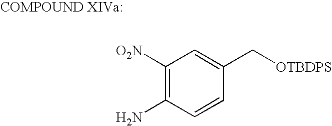

COMPOUND XIa (1 g, 5.10 mmol) was diluted into dry THF (70 mL), cooled to 0° C., and treated with lithium aluminum hydride (250 mg, 6.57 mmol) after which the reaction mixture was maintained at 0° C. for 15 min. The mixture was partitioned between water and ethyl acetate, and the organic phase dried over anhydrous sodium sulfate and concentrated in vacuo. The crude material was then diluted into DMF (30 mL), treated with imidazole (1.6 g, 24 mmol), tert-butyldiphenylsilyl chloride (1.8 g, 12 mmol) and maintained at 23° C. for 15 h. The reaction mixture was partitioned between water and diethyl ether, and the organic phase dried over anhydrous sodium sulfate and concentrated in vacuo. The crude material was purified by flash chromatography (SiO₂, 15% acetone-hexane) to provide COMPOUND XIVa which was characterized by ¹H NMR and HPLC.

### EXAMPLE 45

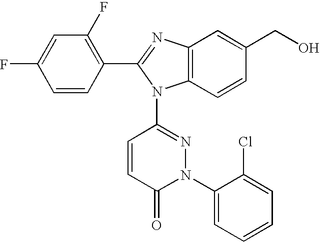

COMPOUND XIVa was manipulated through the same transformations described for the synthesis of EXAMPLE 39 to provide the silyl-protected EXAMPLE 45. The tert-butyldiphenylsilyl ether (400 mg, 0.569 mmol) was diluted into dry THF (15 mL), cooled to 0° C., and treated with a 1M THF solution of tetrabutylammonium fluoride (0.63 mL, 0.626 mmol) after which the reaction mixture was maintained at 0° C. for 2 h. The mixture was partitioned between water and ethyl acetate, and the organic phase dried over anhydrous sodium sulfate and concentrated in vacuo. The crude material was purified by flash chromatography (SiO₂, acetone-hexane) to provide EXAMPLE 45 which was characterized by HPLC and mass spectrometry (m/z: 465 (M⁺+1)).

### EXAMPLE 46

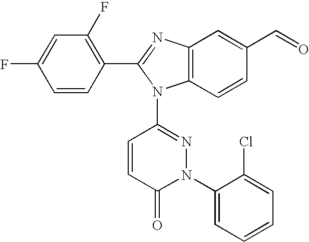

EXAMPLE 45 (25 mg, 0.054 mmol) was diluted into dry CH₂Cl₂ (1 mL) and treated with Dess-Martin reagent (34 mg, 0.081 mmol) at 23° C. under argon. The reaction mixture was maintained at 23° C. for 2 h, partitioned between NaHCO_{3(aq)} and CH₂Cl₂, the organic phase dried over anhydrous sodium sulfate and concentrated in vacuo. The crude product was purified by preparative thin layer chromatography (SiO₂, 2 plates, 1000 micron, 20×20 cm, 5% MeOH—CHCl₃) to provide EXAMPLE 46 which was characterized by ¹H NMR, HPLC and mass spectrometry (m/z: 463 (M⁺+1)).

### EXAMPLE 47

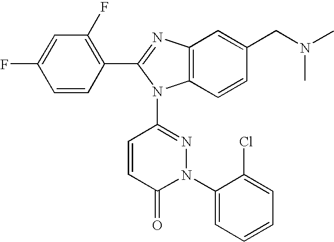

EXAMPLE 46 (19 mg, 0.041 mmol) was diluted into dry CH₂Cl₂ (1 mL), treated with N,N-diisopropylethylamine (0.023 mL, 0.123 mmol), a 2M THF solution of dimethylamine (0.031 mL, 0.062 mmol), sodium triacetoxyborohydride (17 mg, 0.082 mmol), and the reaction mixture was maintained at 23° C. for 15 h. The reaction mixture was then partitioned between NaHCO_{3(aq)} and CH₂Cl₂, the organic phase dried over anhydrous sodium sulfate and concentrated in vacuo to provide EXAMPLE 47 which was characterized by ¹H NMR, HPLC and mass spectrometry (m/z: 492 (M⁺+1)).

### EXAMPLE 48

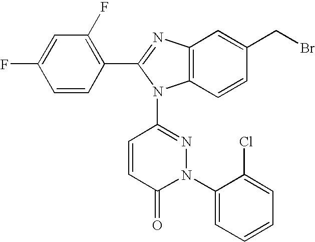

EXAMPLE 45 (50 mg, 0.108 mmol) was diluted into dry CH₂Cl₂ (0.6 mL) and THF (1.1 mL), treated with a 1M CH₂Cl₂ solution of PBr₃ (0.3 mL, 0.323 mmol), and the reaction mixture was maintained at 23° C. for 15 h under argon. The reaction mixture was then partitioned between NaHCO_{3(aq)} and CH₂Cl₂, the organic phase dried over anhydrous sodium sulfate and concentrated in vacuo. The crude product was purified by preparative thin layer chromatography (SiO₂, 4 plates, 1000 micron, 20×20 cm, 30% acetone-hexane) to provide EXAMPLE 48 which was characterized by ¹H NMR, HPLC and mass spectrometry (m/z: 527 (M⁺+1)).

### EXAMPLE 49

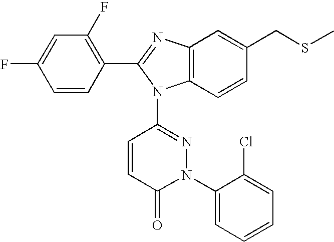

EXAMPLE 48 (38 mg, 0.072 mmol) was diluted into dry CH₂Cl₂ (1.5 mL), treated with NaSMe (15 mg, 0.217 mmol), and the reaction mixture was maintained at 23° C. for 15 h under argon. The reaction mixture was then partitioned between NaHCO_{3(aq)} and CH₂Cl₂, the organic phase dried over anhydrous sodium sulfate and concentrated in vacuo. The crude product was purified by preparative thin layer chromatography (SiO₂, 2 plates, 1000 micron, 20×20 cm, 30% ethyl acetate-hexane) to provide EXAMPLE 49 which was characterized by ¹H NMR, HPLC and mass spectrometry (m/z: 495 (M⁺+1)).

### EXAMPLE 50

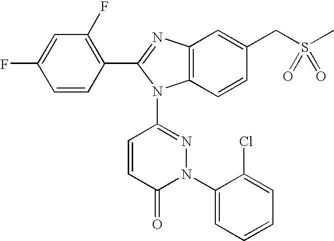

EXAMPLE 49 (21 mg, 0.043 mmol) was diluted into MeOH (1 mL), and treated dropwise with a water solution (0.4 mL) of oxone (56 mg, 0.089 mmol). The reaction mixture was maintained at 23° C. for 2 h, partitioned between water and CH₂Cl₂, the organic phase dried over anhydrous sodium sulfate and concentrated in vacuo. The crude product was purified by preparative thin layer chromatography (SiO₂, 1000 micron, 20×20 cm, 40% acetone-hexane) to provide EXAMPLE 50 which was characterized by ¹H NMR, HPLC and mass spectrometry (m/z: 527 (M⁺+1)).

### EXAMPLE 51

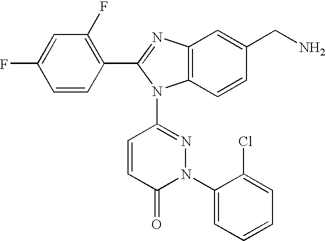

EXAMPLE 51 was prepared from EXAMPLE 45 following the procedure in COMPOUND Va for the generation of the amine from the intermediate alcohol. The purified product was characterized by HPLC and mass spectrometry (m/z: 464 (M⁺+1)).

### EXAMPLE 52

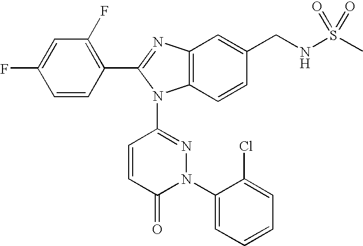

EXAMPLE 52 was prepared from EXAMPLE 51 following the procedure in COMPOUND VIa for the generation of the methylsulfonamide. The purified product was characterized by ¹H NMR, HPLC and mass spectrometry (m/z: 542 (M⁺+1)).

### EXAMPLE 53

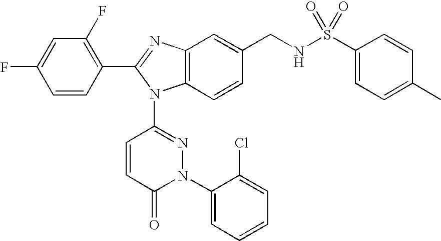

EXAMPLE 53 was prepared from EXAMPLE 51 following the procedure in COMPOUND VIa for the generation of the methylsulfonamide, but substituting toluenesulfonyl chloride for methanesulfonyl chloride. The purified product was characterized by HPLC and mass spectrometry (m/z: 618 (M⁺+1)).

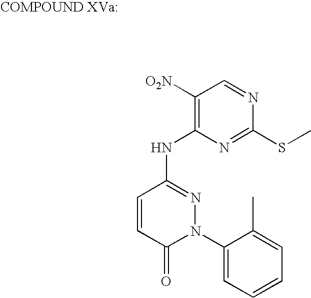

4-Amino-5-nitro-2-methylthiopyrimidine (50 mg, 0.269 mmol), prepared by literature methods known to those skilled in the art, was diluted into DME (2 mL), combined with COMPOUND Xa (110 mg, 0.403 mmol) and degassed via a stream of nitrogen bubbled through the mixture. Cesium carbonate (88 mg, 0.269 mmol), xanthphos (20 mg, 0.035 mmol) and Pd₂(dba)₂ (15 mg, 0.016 mmol) were added sequentially, and the reaction mixture was heated at 95° C. for 15 h under argon. The reaction mixture was filtered through a pad of celite, washed with DME and concentrated in vacuo. The crude product was purified by preparative thin layer chromatography (4 plates, SiO₂, 1000 micron, 20×20 cm, 50% ethyl acetate-hexane) to provide COMPOUND XVa which was characterized by ¹H NMR, HPLC and mass spectrometry (m/z: 371 (M⁺+1)).

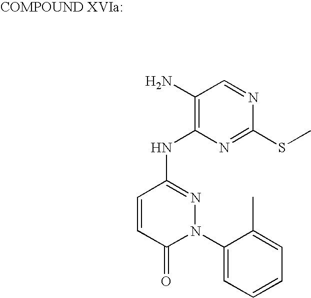

COMPOUND XVa (50 mg, 0.135 mmol) was diluted into CH₂Cl₂ (4 mL), treated with catalytic palladium on carbon, evacuated and flushed with hydrogen gas via a double balloon, and stirred at rt for 14 h under a positive pressure of hydrogen. The reaction mixture was filtered through a pad of celite, washed with methylene chloride and concentrated in vacuo. The crude product was purified by preparative thin layer chromatography (2 plates, SiO₂, 1000 micron, 20×20 cm, 50% ethyl acetate-hexane) to provide COMPOUND XVIa which was characterized by HPLC and mass spectrometry (m/z: 341 (M⁺+1)).

### EXAMPLE 54

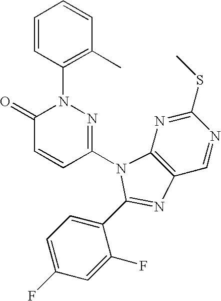

COMPOUND XVIa (16 mg, 0.047 mmol) was diluted into dry nitrobenzene (15 mL), treated with excess molecular sieves, and 2,4-difluorobenzaldehyde (0.007 mL, 0.065 mmol) was added. The reaction mixture was heated in a sealed tube at 170° C. for 15 h, and the nitrobenzene was then distilled away at 110° C. The crude product was purified by preparative thin layer chromatography (2 plates, Sio₂, 1000 micron, 20×20 cm, 10% MeOH—CH₂Cl₂) to provide EXAMPLE 54 which was characterized by HPLC and mass spectrometry (m/z: 463 (M⁺+1)).

### EXAMPLE 55

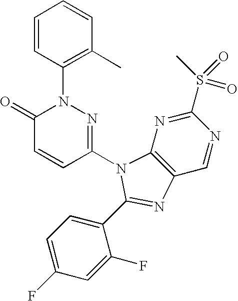

EXAMPLE 54 (7 mg, 0.015 mmol) was diluted into methanol (0.3 mL), treated dropwise with a solution of oxone (18 mg, 0.032 mmol) in water (0.15 mL), and the reaction mixture was stirred at 23° C. for 2 h. The reaction mixture was then partitioned between water and CH₂Cl₂, the organic phase dried over anhydrous sodium sulfate and concentrated in vacuo. The crude product, EXAMPLE 55, was characterized by mass spectrometry (m/z: 495 (M⁺+1)).

### EXAMPLE 56

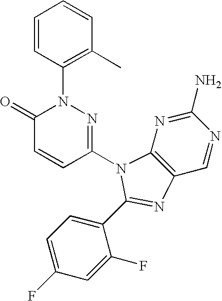

EXAMPLE 55 (3.5 mg, 0.007 mmol) was diluted into DMSO (1 mL), bubbled with ammonia gas for 5 min, and the reaction mixture was heated in a pressure tube at 100° C. for 1.5 h. The DMSO was then distilled away at 100° C. under a stream of nitrogen. The crude residue was purified by preparative thin layer chromatography (SiO₂, b 250 micron, 20×20 cm, 50% ethyl acetate-hexane) to provide EXAMPLE 56 which was characterized by ¹H NMR, HPLC and mass spectrometry (m/z: 432 (M⁺+1)).

### EXAMPLE 57

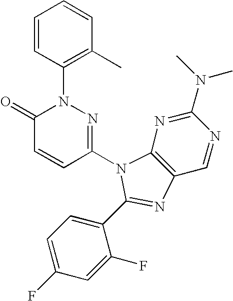

EXAMPLE 57 was prepared as in EXAMPLE 56 by the addition of dimethylamine to sulfone EXAMPLE 55 in DMSO. The purified product was characterized by HPLC and mass spectrometry (m/z: 460 (M⁺+1)).

1. A compound represented by formula (I) or formula (II): 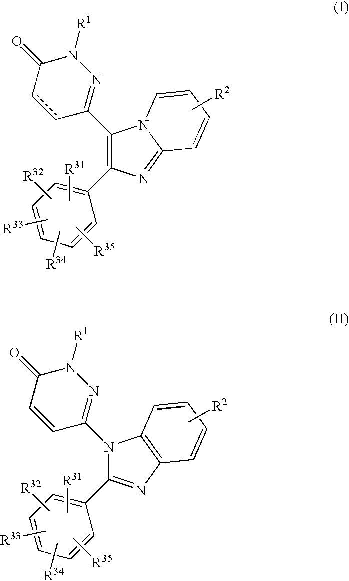
or a pharmaceutically acceptable salt or hydrate thereof, wherein
the dotted line indicates an optional bond;
R¹ is hydrogen, C₁₋₆alkyl- group, C₃₋₆ cycloalkyl- group, aryl group, or arylC₁₋₆alkyl- group, any of the groups optionally substituted with 1-6 substituents, each substituent independently being —OH, —(C₀₋₄alkyl)-N(C₀₋₄alkyl)(C₀₋₄alkyl), C₁₋₄alkyl, C₁₋₆alkoxy, C₁₋₆alkyl-C(O)—C₀₋₄alkyl-, or halogen;
R² is hydrogen, —C(O)—N₃, —NCO, C₁₋₆alkyl- group, —C(O)(C₀₋₄alkyl) group, —(C₀₋₄alkyl)-N(C₀₋₄alkyl)(C₀₋₄alkyl) group, —(C₀₋₄alkyl)-S(O)ₙ—(C₀₋₄alkyl) group, —S(O)₂—N(C₀₋₄alkyl)(C₀₋₄alkyl) group, —C(O)—N(C₀₋₄alkyl)(C₀₋₄alkyl) group, —N(C₀₋₄alkyl)-C(O)—N(C₀₋₄alkyl)(C₀₋₄alkyl) group, —O—C(O—)N(C₀₋₄alkyl)(C₀₋₄alkyl) group, —C(O)—O—C₀₋₄alkyl) group, —C₀₋₆alkyl-N(C₀₋₄alkyl)-S(O)₂—C₀₋₄alkyl) group, or —C₀₋₆alkyl-N(C₀₋₄alkyl)-S(O)₂—(C₀₋₄alkyl)aryl group, any of the groups optionally substituted with 1-6 substituents, each substituent independently being —OH, —N(C₀₋₄alkyl)(C₀₋₄alkyl), C₁₋₄alkyl, C₁₋₆alkoxy, C₁₋₆alkyl-CO—C₀₋₄alkyl-, or halogen;
R³¹, R³², R³³, R³⁴, R³⁵ each independently is hydrogen, halogen, or C₁₋₆alkyl- group optionally substituted with 1-6 substituents, each substituent independently being —OH, —N(C₀₋₄alkyl)(C₀₋₄alkyl), C₁₋₆alkoxy, C₁₋₆alkyl-CO—C₀₋₄alkyl-, or halogen;
n is 0, 1, or2; and
any alkyl is optionally substituted with 1-6 independent halogen.

2. The compound according to claim 1, represented by formula (I) or a pharmaceutically acceptable salt thereof.

3. The compound according to claim 2, 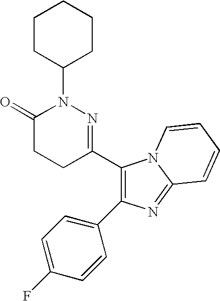 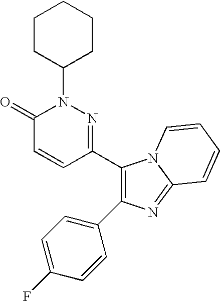 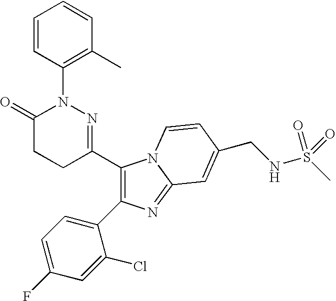
or a pharmaceutically acceptable salt thereof.

4. The compound according to claim 2, represented by

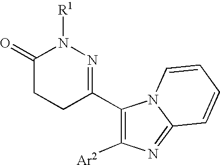

| R¹ Group | Ar² Group |
|---|---|
| 2-Hydroxyethyl | 4-Fluorophenyl |
| 2,2,2-Trifluoroethyl | 4-Fluorophenyl |
| H | 4-Fluorophenyl |
| Benzyl | 4-Fluorophenyl |
| Isopropyl | 4-Fluorophenyl |
| 2-Chlorophenyl | 4-Fluorophenyl |
| 3-Chlorophenyl | 4-Fluorophenyl |
| 2-Chlorophenyl | 2-Chlorophenyl |
| Cyclohexyl | 2,4-Difluorophenyl |
| 2-Chlorophenyl | 3-(Trifluoromethyl)phenyl |
| Cyclohexyl | 3-(Trifluoromethyl)phenyl |
| 2-Chlorophenyl | 2-Chloro-4-fluorophenyl |
| 2,6-Dichlorophenyl | 2-Chloro-4-fluorophenyl |
| 2-Tolyl | 2-Chloro-4-fluorophenyl |
| 2,6-Dichlorophenyl | 2,3-Dichlorophenyl |
| 2-Chlorophenyl | 2,3-Dichlorophenyl |
| 2-Tolyl | 2,3-Dichlorophenyl |
| 2-(Trifluoromethyl) | 2,3-Dichlorophenyl |
| phenyl | |
| 2-Tolyl | 2,4-Difluorophenyl |
| 2,6-Dichlorophenyl | 2,4-Difluorophenyl |

or a pharmaceutically acceptable salt thereof.

5. The compound according to claim 2, represented by

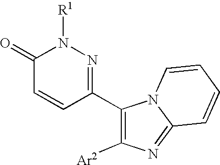

| R¹ Group | Ar² Group |
|---|---|
| 2,2,2-Trifluoroethyl | 4-Fluorophenyl |
| 2,6-Dichlorophenyl | 4-Fluorophenyl |
| 2-Chlorophenyl | 2-Chlorophenyl |
| Cyclohexyl | 2,4-Difluorophenyl |
| 2-Chlorophenyl | 3-(Trifluoromethyl)phenyl |
| Cyclohexyl | 3-(Trifluoromethyl)phenyl |
| 2,6-Dichlorophenyl | 3-(Trifluoromethyl)phenyl |
| 2-Chlorophenyl | 2-Chloro-4-fluorophenyl |
| 2,6-Dichlorophenyl | 2-Chloro-4-fluorophenyl |
| 2-Tolyl | 2-Chloro-4-fluorophenyl |
| 2,6-Dichlorophenyl | 2,3-Dichlorophenyl |
| 2-Tolyl | 2,3-Dichlorophenyl |
| 2-(Trifluoromethyl) | 2,3-Dichlorophenyl |
| phenyl | |
| 2-Tolyl | 2,4-Difluorophenyl |
| 2,6-Dichlorophenyl | 2,4-Difluorophenyl |

or a pharmaceutically acceptable salt thereof.

6. The compound according to claim 1, represented by formula (II) or a pharmaceutically acceptable salt thereof.

7. The compound according to claim 6, represented by 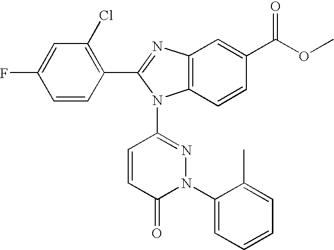 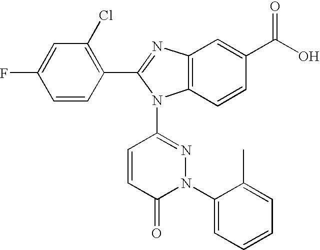 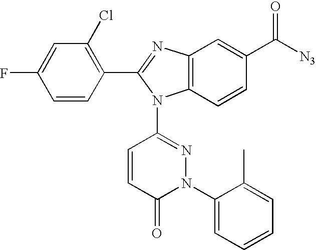 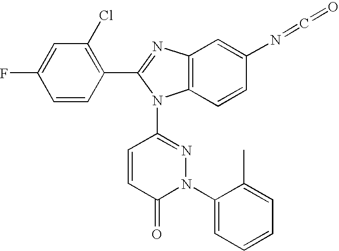 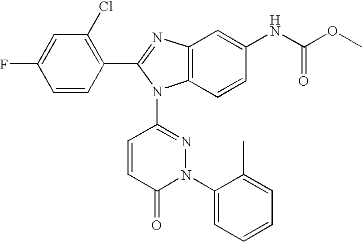 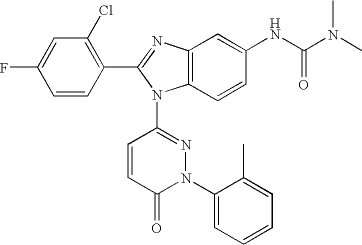 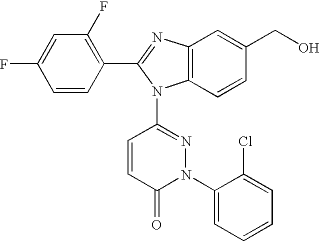 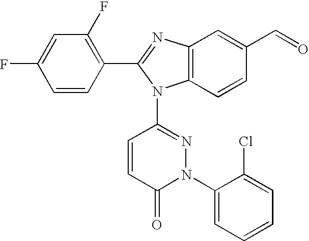 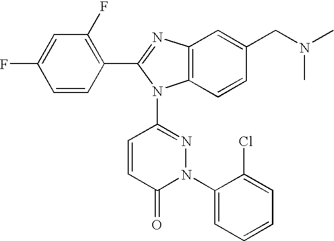 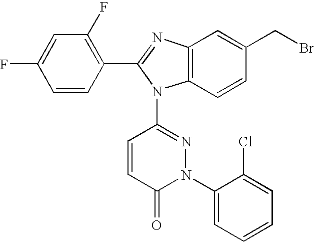 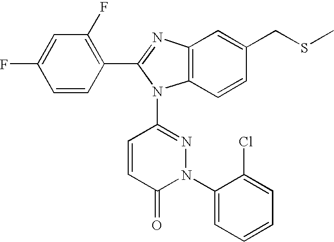 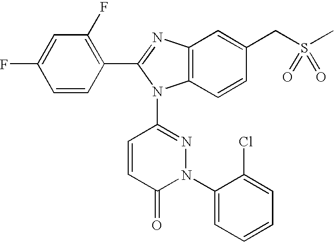 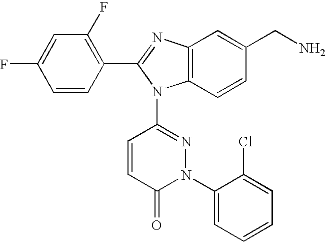 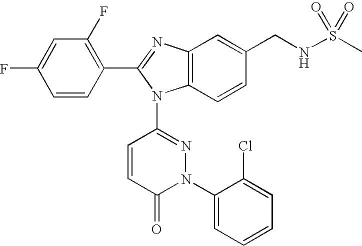 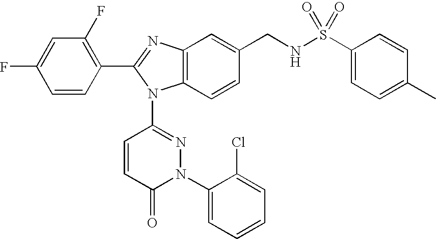 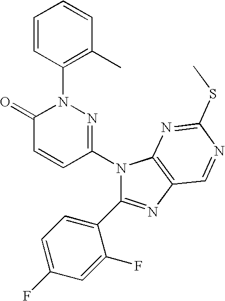 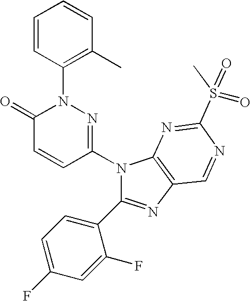 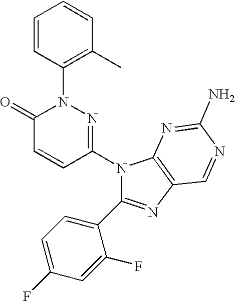 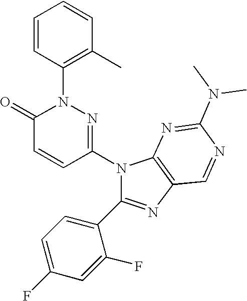
or a pharmaceutically acceptable salt thereof.

8. A pharmaceutical composition comprising a compound in accordance with claim 1 in combination with a pharmaceutically acceptable carrier.

9. A method of treating a inflammation in a mammalian patient in need of such treatment, which is comprised of administering to said patient an anti-inflammatory effective amount of a compound as described in claim 1.

10. A method of treating rheumatoid arthritis, osteoarthritis, endotoxemia, toxic shock syndrome, inflammatory bowel disease, tuberculosis, atherosclerosis, muscle degeneration, cachexia, psoriatic arthritis, Reiter's syndrome, rheumatoid arthritis, gout, traumatic arthritis, rubella arthritis or acute synovitis by administering an effective amount of a compound as described in claim 1.

11. A method of treating rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcosis, bone resorption diseases, reperfusion injury, graft v. host rejection, allograft rejection, fever, myalgia due to infection, cachexia secondary to infection or malignancy, cachexia secondary to acquired immune deficiency syndrome (AIDS), AIDS related complex (ARC), keloid formation, scar tissue formation, Crohn's disease, ulcerative colitis or pyresis by adminstering an effective amount of a compound as described in claim 1.

12. A method of treating osteoporosis in a mammalian patient in need of such treatment, which is comprised of administering to said patient an effective amount of a compound as described in claim 1.

13. A method of treating bone resorption in a mammalian patient in need of such treatment, which is comprised of administering to said patient an effective amount of a compound as described in claim 1.

14. A method of treating Crohn's disease in a mammalian patient in need of such treatment which is comprised of administering to said patient an effective amount of a compound as described in claim 1.

15. A method of treating dementia, neurodegeneraton, or Parkinson's disease in a mammalian patient in need of such treatment which is comprised of administering to said patient an effective amount of a compound as described in claim 1.

16. A method of treating depression, anxiety, psychosis, schizophrenia, or substance abuse in a mammalian patient in need of such treatment which is comprised of administering to said patient an effective amount of a compound as described in claim 1.

17. A method of treating pain or migraine in a mammalian patient in need of such treatment which is comprised of administering to said patient an effective amount of a compound as described in claim 1.

18. A method of treating stroke and cerebrovascular disease in a mammalian patient in need of such treatment which is comprised of administering to said patient an effective amount of a compound as described in claim 1.

19. A process for making a pharmaceutical composition comprising combining a compound of claim 1 and a pharmaceutically acceptable carrier.
